SEQUENCE LISTING (5 sequences)

SEQ ID NO: 1 - protein, 3144 aa, Homo sapiens
MATLEKLMKAFESLKSFQQQQQQQQQQQQQQQQQQQQQQQPPPPPPPPPPPQLPQPPPQAQPLLPQPQPPPPPPPPPPGPAVAEEPLHRPKKELSATKKDRVNHCLTICENIVAQSVRNSPEFQKLLGIAMELFLLCSDDAESDVRMVADECLNKVIKALMDSNLPRLQLELYKEIKKNGAPRSLRAALWRFAELAHLVRPQKCRPYLVNLLPCLTRTSKRPEESVQETLAAAVPKIMASFGNFANDNEIKVLLKAFIANLKSSSPTIRRTAAGSAVSICQHSRRTQYFYSWLLNVLLGLLVPVEDEHSTLLILGVLLTLRYLVPLLQQQVKDTSLKGSFGVTRKEMEVSPSAEQLVQVYELTLHHTQHQDHNVVTGALELLQQLFRTPPPELLQTLTAVGGIGQLTAAKEESGGRSRSGSIVELIAGGGSSCSPVLSRKQKGKVLLGEEEALEDDSESRSDVSSSALTASVKDEISGELAASSGVSTPGSAGHDIITEQPRSQHTLQADSVDLASCDLTSSATDGDEEDILSHSSSQVSAVPSDPAMDLNDGTQASSPISDSSQTTTEGPDSAVTPSDSSEIVLDGTDNQYLGLQIGQPQDEDEEATGILPDEASEAFRNSSMALQQAHLLKNMSHCRQPSDSSVDKFVLRDEATEPGDQENKPCRIKGDIGQSTDDDSAPLVHCVRLLSASFLLTGGKNVLVPDRDVRVSVKALALSCVGAAVALHPESFFSKLYKVPLDTTEYPEEQYVSDILNYIDHGDPQVRGATAILCGTLICSILSRSRFHVGDWMGTIRTLTGNTFSLADCIPLLRKTLKDESSVTCKLACTAVRNCVMSLCSSSYSELGLQLIIDVLTLRNSSYWLVRTELLETLAEIDFRLVSFLEAKAENLHRGAHHYTGLLKLQERVLNNVVIHLLGDEDPRVRHVAAASLIRLVPKLFYKCDQGQADPVVAVARDQSSVYLKLLMHETQPPSHFSVSTITRIYRGYNLLPSITDVTMENNLSRVIAAVSHELITSTTRALTFGCCEALCLLSTAFPVCIWSLGWHCGVPPLSASDESRKSCTVGMATMILTLLSSAWFPLDLSAHQDALILAGNLLAASAPKSLRSSWASEEEANPAATKQEEVWPALGDRALVPMVEQLFSHLLKVINICAHVLDDVAPGPAIKAALPSLTNPPSLSPIRRKGKEKEPGEQASVPLSPKKGSEASAASRQSDTSGPVTTSKSSSLGSFYHLPSYLKLHDVLKATHANYKVTLDLQNSTEKFGGFLRSALDVLSQILELATLQDIGKCVEEILGYLKSCFSREPMMATVCVQQLLKTLFGTNLASQFDGLSSNPSKSQGRAQRLGSSSVRPGLYHYCFMAPYTHFTQALADASLRNMVQAEQENDTSGWFDVLQKVSTQLKTNLTSVTKNRADKNAIHNHIRLFEPLVIKALKQYTTTTCVQLQKQVLDLLAQLVQLRVNYCLLDSDQVFIGFVLKQFEYIEVGQFRESEAIIPNIFFFLVLLSYERYHSKQIIGIPKIIQLCDGIMASGRKAVTHAIPALQPIVHDLFVLRGTNKADAGKELETQKEVVVSMLLRLIQYHQVLEMFILVLQQCHKENEDKWKRLSRQIADIILPMLAKQQMHIDSHEALGVLNTLFEILAPSSLRPVDMLLRSMFVTPNTMASVSTVQLWISGILAILRVLISQSTEDIVLSRIQELSFSPYLISCTVINRLRDGDSTSTLEEHSEGKQIKNLPEETFSRFLLQLVGILLEDIVTKQLKVEMSEQQHTFYCQELGTLLMCLIHIFKSGMFRRITAAATRLFRSDGCGGSFYTLDSLNLRARSMITTHPALVLLWCQILLLVNHTDYRWWAEVQQTPKRHSLSSTKLLSPQMSGEEEDSDLAAKLGMCNREIVRRGALILFCDYVCQNLHDSEHLTWLIVNHIQDLISLSHEPPVQDFISAVHRNSAASGLFIQAIQSRCENLSTPTMLKKTLQCLEGIHLSQSGAVLTLYVDRLLCTPFRVLARMVDILACRRVEMLLAANLQSSMAQLPMEELNRIQEYLQSSGLAQRHQRLYSLLDRFRLSTMQDSLSPSPPVSSHPLDGDGHVSLETVSPDKDWYVHLVKSQCWTRSDSALLEGAELVNRIPAEDMNAFMMNSEFNLSLLAPCLSLGMSEISGGQKSALFEAAREVTLARVSGTVQQLPAVHHVFQPELPAEPAAYWSKLNDLFGDAALYQSLPTLARALAQYLVVVSKLPSHLHLPPEKEKDIVKFVVATLEALSWHLIHEQIPLSLDLQAGLDCCCLALQLPGLWSVVSSTEFVTHACSLIYCVHFILEAVAVQPGEQLLSPERRTNTPKAISEEEEEVDPNTQNPKYITAACEMVAEMVESLQSVLALGHKRNSGVPAFLTPLLRNIIISLARLPLVNSYTRVPPLVWKLGWSPKPGGDFGTAFPEIPVEFLQEKEVFKEFIYRINTLGWTSRTQFEETWATLLGVLVTQPLVMEQEESPPEEDTERTQINVLAVQAITSLVLSAMTVPVAGNPAVSCLEQQPRNKPLKALDTRFGRKLSIIRGIVEQEIQAMVSKRENIATHHLYQAWDPVPSLSPATTGALISHEKLLLQINPERELGSMSYKLGQVSIHSVWLGNSITPLREEEWDEEEEEEADAPAPSSPPTSPVNSRKHRAGVDIHSCSQFLLELYSRWILPSSSARRTPAILISEVVRSLLVVSDLFTERNQFELMYVTLTELRRVHPSEDEILAQYLVPATCKAAAVLGMDKAVAEPVSRLLESTLRSSHLPSRVGALHGVLYVLECDLLDDTAKQLIPVISDYLLSNLKGIAHCVNIHSQQHVLVMCATAFYLIENYPLDVGPEFSASIIQMCGVMLSGSEESTPSIIYHCALRGLERLLLSEQLSRLDAESLVKLSVDRVNVHSPHRAMAALGLMLTCMYTGKEKVSPGRTSDPNPAAPDSESVIVAMERVSVLFDRIRKGFPCEARVVARILPQFLDDFFPPQDIMNKVIGEFLSNQQPYPQFMATVVYKVFQTLHSTGQSSMVRDWVMLSLSNFTQRAPVAMATWSLSCFFVSASTSPWVAAILPHVISRMGKLEQVDVNLFCLVATDFYRHQIEEELDRRAFQSVLEVVAAPGSPYHRLLTCLRNVHKVTTC

SEQ ID NO: 2 - RNA, 21 nt, artificial sequence
  siRNA
aacuuucagcuaccaagaaag

SEQ ID NO: 3 - RNA, 21 nt, artificial sequence
  siRNA
cuuucuugguagcugaaaguu

SEQ ID NO: 4 - DNA, 46 bp, Artificial sequence
  Huntingtin gene subsequence
agctttgatggattctaatctcttgaaattagaatccatcaaagct

SEQ ID NO: 5 - RNA, 21 nt, Artificial sequence
  siRNA control sequence
gauagcaaugacgaaugcgua

(19) United States
(12) Patent Application Publication
(10) Pub. No.: US 2011/0039789 A1
(43) Pub. Date: Feb. 17, 2011

(54) Use of Huntingtin Protein for the Diagnosis and the Treatment of Cancer

(75) Inventors: Sandrine Humbert, Orsay (FR); Frederic Saudou, Orsay (FR); John McGuire, Issy Les Moulineaux (FR); Anne Vincent Salomon, Paris (FR)

Correspondence Address: SALIWANCHIK LLOYD & SALIWANCHIK;A PROFESSIONAL ASSOCIATION, PO Box 142950, GAINESVILLE, FL, 32614, US

(73) Assignees: INSTITUT CURIE, Paris Cedex 05 (FR); CENTRE NATIONAL DE LA RECHERCHE SCIENTIFIQUE (CNRS), Paris (FR)

(21) Appl. No.: 12/855,735

(22) Filed: Aug. 13, 2010

Related U.S. Application Data
(60) Provisional application No. 61/233,864, filed on Aug. 14, 2009.

(51) Int. Cl. A61K 38/02; G01N 33/574; C12Q 1/48; A61K 31/7088; A61K 31/7105; A61P 35/00; A61P 35/02

(52) U.S. Cl. 514/19.4; 435/7.23; 435/15; 514/44 A; 514/44 R; 514/19.3; 514/19.5; 514/19.6

(57) ABSTRACT

The present invention relates to new methods of treatment of cancer, in particular of breast cancer, and methods of screening of compounds useful in the treatment of cancer. The present invention further provides new prognostic and/or diagnostic markers in human cancer.

### CROSS-REFERENCE TO RELATED APPLICATION

This application claims the benefit of U.S. Provisional Application No. 61/233,864, filed Aug. 14, 2009, which is incorporated herein by reference.

### FIELD OF THE INVENTION

The present invention relates to the field of medicine, in particular of oncology. It relates to new methods of treatment of cancer and methods of screening of compounds useful in the treatment of cancer. The present invention further provides new prognostic and/or diagnostic markers in human cancer.

### BACKGROUND OF THE INVENTION

Cancer occurs when cell division gets out of control and results from impairment of a DNA repair pathway, the transformation of a normal gene into an oncogene or the malfunction of a tumor supressor gene. Many different forms of cancer exist. The incidence of these cancers varies but it represents the second highest cause of mortality, after heart disease, in most developed countries. While different forms of cancer have different properties, one factor which many cancers share is the ability to metastasize. Distant metastasis of all malignant tumors remains the primary cause of death in patients with the disease.

Breast cancer is a significant health problem for women in the United States and throughout the world. Although advances have been made in detection and treatment of the disease, breast cancer remains the leading cause of cancer death in women due to recurrence of local and distant metastasis and approximately 40% of the treated patients relapse and ultimately die of metastatic breast cancer.

No universally successful method for the prevention or treatment of breast cancer is available and the management of the disease currently relies on an early diagnosis and aggressive treatment, which may include surgery, radiotherapy, chemotherapy and hormone therapy. However, the high mortality observed in breast cancer patients indicates that improvements are needed in the treatment, diagnosis and prevention of this disease.

Prostate cancer is the most common cancer and the second leading cause of cancer-related deaths among men in North America and Europe. Prompt detection and treatment is needed to limit mortality caused by prostate cancer. Localized prostate tumours are commonly diagnosed and conventionally treated by radical prostatectomy. Overwhelming clinical evidence shows that human prostate cancer has the propensity to metastasize to bone, and the disease appears to progress inevitably from androgen dependent to androgen refractory status, leading to increased patient mortality.

In spite of considerable research into therapies for the disease, prostate cancer remains difficult to diagnose and treat effectively. Accordingly, there is a need in the art for improved methods for detecting and treating such cancers.

### SUMMARY OF THE INVENTION

The inventors surprisingly demonstrate herein that huntingtin (htt) protein, in particular the phosphorylated form of this protein, has a protective effect against the progression of cancer.

Accordingly, in a first aspect, the present invention concerns a method for treating a cancer in a subject, the method comprising administering a therapeutically effective amount of a compound increasing the cellular level of the phosphorylated form of huntingtin.

Preferably, the huntingtin protein is phosphorylated at one or several positions selected from the group consisting of S421, S535, S1181, S1201, S2076, S2653 and S2657. More preferably, the huntingtin protein is phosphorylated at position S421.

In an embodiment, the compound increasing the cellular level of the phosphorylated form of huntingtin inhibits the dephosphorylation of huntingtin. Preferably, said compound is a calcineurin inhibitor or a compound inhibiting the interaction between calcineurin and huntingtin. The calcineurin inhibitor may be selected from the group consisting of FK506, cyclosporin A, FK520, L685,818, FK523, 15-0-DeMe-FK-520, Lie120, fenvalerate, resmethrin, cypermethrin, deltamethrin and an analogue thereof. Alternatively, the calcineurin inhibitor may be a nucleic acid molecule interfering specifically with calcineurin expression, preferably a RNAi, an antisense nucleic acid or a ribozyme. The calcineurin inhibitor may also be a dominant-interfering form of calcineurin.

In another embodiment, the compound increasing the cellular level of the phosphorylated form of huntingtin is selected from the group consisting of huntingtin protein, huntingtin protein comprising the mutation S241D and a biologically active fragment thereof, and a nucleic acid encoding them.

In a further embodiment, the compound increasing the cellular level of the phosphorylated form of huntingtin increases the phosphorylation of huntingtin. Preferably, said compound increases the activity of the kinase Akt, protein kinase A, Polo kinase 1, AuroraA and AuroraB and/or SGK.

Preferably, the cancer to be treated is an invasive cancer and/or a cancer capable of metastasis. More preferably, the cancer is selected from the group consisting of leukemia, lymphoma, melanoma, lung cancer, bowel cancer, colon cancer, rectal cancer, colorectal cancer, brain cancer, liver cancer, pancreatic cancer, breast cancer, prostate cancer, testicular cancer and retinoblastoma. Even more preferably, the cancer is breast cancer or prostate cancer.

In a preferred embodiment, the subject is a human. In a particular embodiment, the subject is a human not affected with Huntington's disease.

In a second aspect, the present invention concerns a method for diagnosing or detecting a cancer in a subject, wherein the method comprises the step of determining the cellular level of phosphorylated form of huntingtin in a sample from said subject, a low cellular level of phosphorylated huntingtin indicating that said subject suffers from a cancer. In an embodiment, the method further comprises the step of comparing the cellular level of phosphorylated huntingtin to a reference cellular level, preferably to the cellular level of phosphorylated huntingtin in a normal sample.

In a third aspect, the present invention concerns a method for predicting, prognosing or monitoring clinical outcome of a subject affected with a cancer, wherein the method comprises the step of determining the expression level of huntingtin in a cancer sample from said subject, a low expression level of huntingtin being indicative of a poor prognosis. Preferably, the method further comprises the step of comparing the expression level of huntingtin to a reference expression level.

In a fourth aspect, the present invention concerns a method for predicting, prognosing or monitoring clinical outcome of a subject affected with a cancer, wherein the method comprises the step of determining the cellular level of phosphorylated huntingtin in a cancer sample from said subject, a low cellular level of phosphorylated huntingtin being indicative of a poor prognosis. Preferably, the method further comprises the step of comparing the cellular level of phosphorylated huntingtin to a reference cellular level.

In another aspect, the present invention concerns a method for predicting, prognosing or monitoring clinical outcome of a subject affected with a cancer, wherein the method comprises the step of determining the number of glutamine residues on the poly-Q expansion of huntingtin in a sample from said subject, a poly-Q expansion comprising more than 20 glutamine residues being indicative of a poor prognosis. In an embodiment, a poly-Q expansion comprising more than 35 glutamine residues is indicative of a poor prognosis. In another embodiment, a poly-Q expansion comprising more than 40 glutamine residues is indicative of a poor prognosis. In an embodiment, the sample from the subject is a cancer sample.

In further aspect, the present invention concerns a method for selecting a subject affected with a cancer for an antitumoral therapy, preferably an adjuvant chemotherapy and/or radiotherapy or determining whether a subject affected with a cancer is susceptible to benefit from an antitumoral therapy, preferably an adjuvant chemotherapy and/or radiotherapy, wherein the method comprises the step of determining the cellular level of phosphorylated huntingtin in a cancer sample from said subject, a low cellular level of phosphorylated huntingtin indicating that an antitumoral therapy, preferably an adjuvant chemotherapy and/or radiotherapy, is required.

In another aspect, the present invention concerns a method for selecting a subject affected with a cancer for an antitumoral therapy, preferably an adjuvant chemotherapy and/or radiotherapy, or determining whether a subject affected with a cancer is susceptible to benefit from an antitumoral therapy, preferably an adjuvant chemotherapy and/or radiotherapy, wherein the method comprises the step of determining the expression level of huntingtin in a cancer sample from said subject as described above, a low expression level of huntingtin indicating that an antitumoral therapy, preferably an adjuvant chemotherapy and/or radiotherapy, is required.

In another aspect, the present invention concerns a method for selecting a subject affected with a cancer for an antitumoral therapy, preferably an adjuvant chemotherapy and/or radiotherapy, or determining whether a subject affected with a cancer is susceptible to benefit from an antitumoral therapy, preferably an adjuvant chemotherapy and/or radiotherapy, wherein the method comprises the step of determining the number of glutamine residues on the poly-Q expansion of huntingtin in a sample from said subject as described above, a poly-Q expansion comprising more than 20 glutamine residues indicating that an antitumoral therapy, preferably an adjuvant chemotherapy and/or radiotherapy, is required. In an embodiment, the sample from the subject is a cancer sample.

In a last aspect, the present invention concerns a method for selecting, identifying or screening a compound useful for treating a subject having cancer, comprising the selection or identification of a compound capable of increasing the expression level and/or the phosphorylation of huntingtin.

In an embodiment, the method comprises:

a) providing a huntingtin protein or a fragment thereof of at least 50 consecutive amino acids and comprising at least one phosphorylated residue selected from the group consisting of S421, S535, S1181, S1201, S2076, S2653 and S2657;

b) providing a compound dephosphorylating at least one phosphorylated residue comprised in htt protein or the fragment thereof provided in step a);

c) contacting a candidate compound with said huntingtin protein or fragment thereof and said dephosphorylating compound; and,

d) selecting the candidate compound that inhibits the dephosphorylation of at least one phosphorylated residue comprised in htt protein or the fragment thereof provided in step a) by dephosphorylating compound.

In another embodiment, the method comprises:

a) contacting a candidate compound with a cell expressing a huntingtin protein and comprising a kinase which phosphorylates huntingtin at a position selected from the group consisting of S421, S535, S1181, S1201, S2076, S2653 and S2657, and a compound dephosphorylating the phosphorylated residue at selected position;

b) assessing the amount of huntingtin phosphorylated and/or the amount of huntingtin which is not phosphorylated; and,

c) selecting the candidate compound that increases the phosphorylation of huntingtin at selected position in comparison with a control cell which has not been contacted with the candidate compound.

In a further embodiment, the method comprises:

a) contacting a candidate compound with a cell expressing a huntingtin protein;

b) assessing the amount of huntingtin expressed in said cell; and

c) selecting the candidate compound that increases the expression of huntingtin in comparison with a control cell which has not been contacted with the candidate compound.

### BRIEF DESCRIPTION OF THE DRAWINGS

FIGS. 1I-1J. Htt phosphorylation is lost in invasive mammary cancer cells. A, Upper panel, western blot analysis of four mammary cell lines and two neuronal cells lines. Lower panel, coomassie blue staining to demonstrate total protein levels. B, Upper panel, western blot analysis of normal and tumor samples from two cases of ductal carcinomas and two cases of lobular carcinomas, showing higher levels in tumors than in normal cells. Lower panel, coomassie blue staining. C, E, G, Total Htt staining of healthy mammary lobules, an in situ tumor, and invasive cancer cells, respectively, from human biopsy samples. D, F, H, Phospho-S421-Htt staining of healthy mammary lobules, an in situ tumor, and invasive cancer cells, respectively, from the same patient as in C, E, and G (scale bar in H 20 μm). I and J, Quantification of htt phosphorylation in invasive ductal and lobular cancers, respectively, from human biopsy samples. Only samples that contained positively stained residual healthy structures were included in the analysis.

FIGS. 2A-2E. Htt regulates cell motility. A, Confocal images of 2a1 cells expressing shLuc (upper), shHtt (middle), or shHtt plus an shRNA-resistant CTFL-Htt. The shRNA constructs express GFP from a separate promoter, allowing identification of transfected cells. The right column shows that the addition of CTFL-Htt rescues the shHtt-induced decrease in total htt to the endogenous levels in shLuc-expressing cells (compare lower right panel to upper right panel). B and C, Scratch closure assays in MCF7 and 4T1 cells, respectively, showing faster closure in cells that were depleted of endogenous htt than in controls. D, Random migration assays of shLuc- and shHtt-expressing MCF7 cells. E, Random migration assays of 2a1 transfected with shLuc+cherry, shHtt+cherry, shHtt+CTFLHtt-WT, shHtt+CTFLHtt-S421A, and shHtt+CTFLHtt-S421D, showing that the unsphosphorylatable S421A mutation does not rescue the increased velocity induced by the depletion of endogenous htt, while the S421-WT and S421D forms do.

FIGS. 3A-3E. Htt controls metastasis and invasion. A, Western blot analysis showing the efficiency of siHtt in 4T1 cells in vitro. B and C, Bioluminescence images merged to photos of Balb/C mice injected with 4t1 cells and treated with seven days of either a scramble RNA (sc) or siHtt (quantified in C, n=11 per group, p<0.01). D, Survival curve of the same sc- and siHtt-treated mice from B and C, showing a significant decrease in the survival of siHtt-treated mice by Kaplan-Meier analysis (p=0.012). E, Images of the lungs from sc- (top) and siHtt- (bottom) treated mice. The three left-most columns show (from left to right) the luciferase, total htt, and nuclear images, obtained by confocal microscopy. The right-most column shows images of whole lungs dissected from mice at the time of death. F, Matrigel Invasion analysis of 4T1 cells stably expressing sc or siHtt without or with different CTFLHtt constructs. The siHtt+CTFLS421A cells showed significantly higher invasion (p<0.001 for all cases) than any of the other groups. No other significant differences were observed.

FIGS. 4A-4B. Oncogene-induced mammary tumors develop faster in HD mouse model. (A) MMTV-PyVT and MMTV-ErbB2 mice were crossed with the Hdh^{Q111/Q111} mouse line carrying a 111 CAG expansion inserted into the endogenous mouse huntingtin gene. Tumor appearance was followed as a function of time (days). WT: MMTV-ErbB2/PyVT; Hdh^{Q7/Q7}; Q111/Q111: MMTV-ErbB2/PyVT; Hdh^{Q111/Q111}. (B) Whole mount of mammary glands were stained with carmine aluminium. Images were acquired with a stereo microscope (0.63×).

FIG. 5. PolyQ-huntingtin does not change tumor growth. Primary solid-tumor isolates from MMTV-PyVT; Hdh^{Q111/Q111} and MMTV-PyVT; Hdh^{Q7/Q7} (WT) were transplanted in immunodeficient mice and tumor size was measured as a function of time following transplantation (days).

FIGS. 6A-6C. PolyQ-huntingtin leads to increased cell motility, invasiveness and metastasis. (A) Random cell migration and (B) matrigel assays were performed with primary cells derived from MMTV-pyVT; Hdh^{Q7/Q7} and MMTV-PyVT; Hdh^{Q111/Q111} tumors. (C) Lungs from immunodeficient mice grafted with primary solid-tumor isolates from MMTV-ErbB2; Hdh^{Q111/Q111} and MMTV-ErbB2; Hdh^{Q7/Q7} were dissected, sectioned and stained with hematoxylin and eosin.

FIG. 7. Breast cancer onset is correlated with the CAG length in HD patients. Nine HD patients developing breast cancer were identified. The length of their abnormal CAG expansion is represented as a function of their age of HD and breast cancer onsets.

### DETAILED DESCRIPTION OF THE INVENTION

Huntingtin (htt) is a large protein whose function and regulation have not been well defined. Expansion of a polyglutamine repeat within the huntingtin protein is known to be the causative event of Huntington's disease (HD), a progressive neurological disorder that leads to a distinctive chorea, cognitive loss, various psychological disorders, and eventually death. As the mutation that causes HD has been known for almost 15 years, the majority of researches have focused on htt during disease progression instead of on the role of the wild-type htt protein. As a consequence, very few elements are known about the role of wild-type htt.

Studies on the cellular localization of the mRNA and protein have revealed that wild-type and polyQ-huntingtin are expressed throughout the nervous system as well as in non-neuronal cells (Sharp and Ross, 1996) suggesting that the wild-type protein's main function may not necessarily be brain-specific.

Some other studies demonstrated an ambiguous correlation between htt and cancer. In 1999, it was suggested that the lower incidence of cancer among HD patients could be due to the polyQ-huntingtin, encountered in HD, which would be able to protect HD patient against cancer by inducing or increasing the rate of naturally occurring programmed cell death in preneoplastic cells (Sorensen et al., 1999). This theory was further supported by two studies demonstrating that the polyglutamine expansion is responsible for cell death induction and that this cell death is mediated by caspase (Saudou et al., 1998; Martindale et al., 1998; Wang et al., 1999). In addition, it was also demonstrated that wild-type htt may be an anti-apoptotic protein (Zeitlin et al., 1995; Rigamonti et al., 2000; Leavitt et al., 2001) and the US patent application US 2002-0187931 describes the use of antagonists to huntingtin protein to treat conditions characterized by dysregulation of cellular proliferation such as cancer.

On the contrary, the inventors herein surprisingly show that wild-type htt, and in particular the phosphorylated form of htt, can be efficiently used in the treatment of cancer. As described in the experimental section, the inventors demonstrate that the decrease of cellular level of the phosphorylated form of htt is associated with the increase of cell motility, invasive and metastasis capacities of cancer cells. Furthermore, they show that the administration of htt or of the phosphorylated form of htt completely rescues the increased motility induced by the knock-down of endogenous htt.

Definitions

As used herein, the term “huntingtin”, “huntingtin protein”, or “htt” refers to the huntingtin protein encoded by the huntingtin gene also called HTT gene, HD gene or IT15 gene (GeneID: 3064). There are many polymorphisms of this gene due to a variable number of CAG codon repeats, encoding glutamine, in the first exon. In its wild-type form, i.e. htt form not causing Huntington's disease, this protein contains from 6 to 35 glutamine residues. In individuals affected by HD, this protein contains more than 35 glutamine residues and is named polyQ-htt. The NCBI accession number of wild-type htt is NP_{—}002102 (SEQ ID NO: 1). The wild-type form of htt has a mass about 350 kD and a size of about 3144 amino acids. Unless otherwise specified, the term “htt”, as used herein, refers to the wild-type form of the htt protein, i.e. htt containing a polyglutamine tract of less than 36 glutamine residues. Htt may be phosphorylated at one or several positions selected from the group consisting of S421, S535, S1181, S1201, S2076, S2653 and S2657. These positions refer to the positions of amino acids in the NCBI sequence of wild-type htt with the accession number NP_{—}002102 (sequence of SEQ ID NO: 1).

The term “cancer” or “tumor”, as used herein, refers to the presence of cells possessing characteristics typical of cancer-causing cells, such as uncontrolled proliferation, immortality, metastatic potential, rapid growth and proliferation rate, and certain characteristic morphological features. This term refers to any type of malignancy (primary or metastases). Typical cancers are solid or hematopoietic cancers such as breast, stomach, oesophageal, sarcoma, ovarian, endometrium, bladder, cervix uteri, rectum, colon, lung or ORL cancers, paediatric tumours (neuroblastoma, glioblastoma multiforme), lymphoma, leukaemia, myeloma, seminoma, Hodgkin and malignant hemopathies. Preferably, the cancer to be treated is an invasive cancer and/or a cancer capable of metastasis. In a particular embodiment, the cancer is selected from the group consisting of leukemia, lymphoma, melanoma, lung cancer, bowel cancer, colon cancer, rectal cancer, colorectal cancer, brain cancer, liver cancer, pancreatic cancer, breast cancer, prostate cancer, testicular cancer and retinoblastoma. In a preferred embodiment, the cancer is a solid cancer, preferably a breast cancer or a prostate cancer, more preferably a breast cancer. Preferably the cancer is an early stage cancer without local or systemic invasion, more preferably an early stage breast cancer without local or systemic invasion.

As used herein, the term “treatment of cancer” refers to any act intended to extend life span of patients such as therapy and retardation of the disease. The treatment can be designed to eradicate the tumor, to stop the progression of the tumor, to prevent the occurrence of metastasis, to promote the regression of the tumor and/or to prevent muscle invasion of cancer. Preferably, the term “treatment of cancer” as used herein, refers to the prevention or delay of metastasis formation, disease progression and/or muscle invasion. The term “therapy” or “antitumoral therapy”, as used herein, refers to any type of treatment of cancer, including an adjuvant therapy and a neoadjuvant therapy. Therapy comprises radiotherapy and therapies, preferably systemic therapies such as hormone therapy, chemotherapy, immunotherapy and monoclonal antibody therapy.

As used herein, the term “chemotherapy” refers to a cancer therapeutic treatment using chemical or biochemical substances, in particular using one or several antineoplastic agents.

The term “radiotherapy” is a term commonly used in the art to refer to multiple types of radiation therapy including internal and external radiation therapies or radioimmunotherapy, and the use of various types of radiations including X-rays, gamma rays, alpha particles, beta particles, photons, electrons, neutrons, radioisotopes, and other forms of ionizing radiations.

The term “immunotherapy” refers to a cancer therapeutic treatment using the immune system to reject cancer. The therapeutic treatment stimulates the patient's immune system to attack the malignant tumor cells. It includes immunization of the patient with tumoral antigens (e.g. by administering a cancer vaccine), in which case the patient's own immune system is trained to recognize tumor cells as targets to be destroyed, or administration of molecules stimulating the immune system such as cytokines, or administration of therapeutic antibodies as drugs, in which case the patient's immune system is recruited to destroy tumor cells by the therapeutic antibodies. In particular, antibodies are directed against specific antigens such as the unusual antigens that are presented on the surfaces of tumors. As illustrating example, one can cite Trastuzumab or Herceptin antibody which is directed against HER2 and approved by FDA for treating breast cancer.

The term “monoclonal antibody therapy” refers to any antibody that functions to deplete tumor cells in a patient. In particular, therapeutic antibodies specifically bind to antigens present on the surface of the tumor cells, e.g. tumor specific antigens present predominantly or exclusively on tumor cells. Alternatively, therapeutic antibodies may also prevent tumor growth by blocking specific cell receptors.

The term “hormone therapy” or “hormonal therapy” refers to a cancer treatment having for purpose to block, add or remove hormones. For instance, in breast cancer, the female hormones estrogen and progesterone can promote the growth of some breast cancer cells. So in these patients, hormone therapy is given to block estrogen and a non-exhaustive list commonly used drugs includes: Tamoxifen, Fareston, Arimidex, Aromasin, Femara, Zoladex/Lupron, Megace, and Halotestin.

The term “adjuvant therapy”, as used herein, refers to any type of treatment of cancer (e.g., chemotherapy or radiotherapy) given as additional treatment, usually after surgical resection of the primary tumor, in a patient affected with a cancer that is at risk of metastasizing and/or likely to recur. The aim of such an adjuvant treatment is to improve the prognosis. Adjuvant therapies comprise radiotherapy and therapy, preferably systemic therapy, such as hormone therapy, chemotherapy, immunotherapy and monoclonal antibody therapy.

The term “neoadjuvant therapy”, as used herein, refers to any type of treatment of cancer given prior to surgical resection of the primary tumor, in a patient affected with a cancer. The most common reason for neoadjuvant therapy is to reduce the size of the tumor so as to facilitate a more effective surgery. Neoadjuvant therapies comprise radiotherapy and therapy, preferably systemic therapy, such as hormone therapy, chemotherapy, immunotherapy and monoclonal antibody therapy.

The term “therapeutically effective amount” refers to that amount of a therapy which is sufficient to reduce or ameliorate the severity, duration and/or progression of a disease or one or more symptoms thereof. As used herein, this term refers to that amount of a compound increasing the cellular level of the phosphorylated form of huntingtin, which is sufficient to destroy, modify, control or remove primary, regional or metastatic cancer tissue, ameliorate cancer or one or more symptoms thereof, or prevent the advancement of cancer, cause regression of cancer, or enhance or improve the therapeutic effect (s) of another therapy (e.g., a therapeutic agent). This term may also refer to the amount of a compound of the present invention sufficient to delay or minimize the spread of cancer or sufficient to provide a therapeutic benefit in the treatment or management of cancer. Further, a therapeutically effective amount with respect to a compound of the present invention means that amount of a compound of the present invention alone, or in combination with other therapeutic agent, that provides a therapeutic benefit in the treatment or management of cancer.

As used herein, the term “poor prognosis” refers to a decreased patient survival and/or an early disease progression and/or an increased cancer invasion and/or an increased metastasis formation.

As used herein, the term “subject” or “patient” refers to an animal, preferably to a mammal, even more preferably to a human, including adult, child and human at the prenatal stage. However, the term “subject” can also refer to non-human animals, in particular mammals such as dogs, cats, horses, cows, pigs, sheep and non-human primates, among others, that are in need of treatment.

The term “sample”, as used herein, means any sample containing cells derived from a subject. Examples of such samples include fluids such as blood, plasma, saliva, urine and seminal fluid samples as well as biopsies, organs, tissues or cell samples. The sample may be treated prior to its use. The term “cancer sample” refers to any sample containing tumoral cells derived from a patient. The term “normal sample” refers to any sample which does not contain any tumoral cells.

The methods of the invention as disclosed below, may be in vivo, ex vivo or in vitro methods, preferably in vitro methods.

As demonstrated in the experimental section, the inventors show that huntingtin (htt) protein, in particular the phosphorylated form of this protein, is involved in the progression of cancer and that an increase of the cellular level of the phosphorylated form of htt allows to prevent cancer invasion and metastasis.

Accordingly, in a first aspect, the present invention concerns a method for treating cancer in a subject, comprising administering a therapeutically effective amount of a compound increasing the cellular level of the phosphorylated form of huntingtin. Said compound may inhibit the dephosphorylation of htt, increase the phosphorylation of htt or increase the cellular level of wild-type htt.

In particular, the phosphorylated htt may be phosphorylated at one or several positions selected from the group consisting of S421, S535, S1181, S1201, S2076, S2653 and S2657. Preferably, the phosphorylated htt is phosphorylated at least at position S421.

In a first embodiment, the compound of the invention inhibits the dephosphorylation of htt. Preferably, the compound inhibits the dephosphorylation of htt at position S421. More preferably, the compound is a calcineurin inhibitor or a compound inhibiting the interaction between calcineurin and huntingtin.

In a preferred embodiment, the compound inhibiting the dephosphorylation of htt is a calcineurin inhibitor. Calcineurin inhibitors include, but are not limited to, cyclosporin A (Novartis International AG, Switzerland), FK506 (Fujisawa Healthcare, Inc., Deerfield, Ill., USA), FK520 (Merck & Co, Rathway, N.J., USA), L685,818 (Merck & Co), FK523, 15-0-DeMe-FK-520 (Liu, Biochemistry, 31:3896-3902 (1992)), Lie120, fenvalerate (Merck & Co), resmethrin (Merck & Co), cypermethrin (Merck & Co) and deltamethrin (Merck & Co), and analogues thereof. WO2005087798 describes cyclosporine derivative inhibiting calcineurin. In a particularly preferred embodiment, the compound inhibiting the dephosphorylation of htt is selected from FK506, cypermethrin, deltamethrin and an analogue thereof. The term “analogue”, as used herein, refers to a compound having similar structural features and having the same biological activity, in particular which inhibits calcineurin.

Calcineurin is a heterodimer composed of a catalytic subunit (Calcineurin A, CaNA) and a regulator subunit (Calcineurin B, CaNB). Then, the activity of calcineurin can also be inhibited by blocking its expression, in particular the expression of one of its subunit. In an embodiment, the activity of calcineurin is inhibited by blocking the expression of the catalytic subunit A, the α (GeneID: 5530), β (GeneID: 5532) or γ (GeneID: 5533) isoform. In another embodiment, the activity of calcineurin is inhibited by blocking the expression of the regulator subunit B, either the α isofom (GeneID: 5534) or the β isoform (GeneID: 5535) or both. The expression of calcineurin can be blocked by any mean known by one skilled in the art.

In a particular embodiment, the calcineurin inhibitor is a nucleic acid molecule interfering specifically with calcineurin expression. Preferably, this nucleic acid is selected from the group consisting of a RNAi, an antisense nucleic acid or a ribozyme.

The term “RNAi” or “interfering RNA” means any RNA which is capable of down-regulating the expression of the targeted protein. It encompasses small interfering RNA (siRNA), double-stranded RNA (dsRNA), single-stranded RNA (ssRNA), micro-RNA (miRNA), and short hairpin RNA (shRNA) molecules. RNA interference, designate a phenomenon by which dsRNA specifically suppresses expression of a target gene at post-translational level. In normal conditions, RNA interference is initiated by double-stranded RNA molecules (dsRNA) of several thousands of base pair length. In vivo, dsRNA introduced into a cell is cleaved into a mixture of short dsRNA molecules called siRNA. The enzyme that catalyzes the cleavage, Dicer, is an endo-RNase that contains RNase III domains (Bernstein et al. 2001). In mammalian cells, the siRNAs produced by Dicer are 21-23 bp in length, with a 19 or 20 nucleotides duplex sequence, two-nucleotide 3′ overhangs and 5′-triphosphate extremities (Zamore et al. 2000; Elbashir et al. 2001). A number of patents and patent applications have described, in general terms, the use of siRNA molecules to inhibit gene expression, for example, WO 99/32619, US 20040053876, US 20040102408 and WO 2004/007718.

siRNA are usually designed against a region 50-100 nucleotides downstream the translation initiator codon, whereas 5′UTR (untranslated region) and 3′UTR are usually avoided. The chosen siRNA target sequence should be subjected to a BLAST search against EST database to ensure that the only desired gene is targeted. Various products are commercially available to aid in the preparation and use of siRNA. In a preferred embodiment, the RNAi molecule is a siRNA of at least about 15-50 nucleotides in length, preferably about 20-30 base nucleotides. For instance, convenient siRNA nucleotides may present the sequences of SEQ ID NOs: 2 and 3.

RNAi can comprise naturally occurring RNA, synthetic RNA, or recombinantly produced RNA, as well as altered RNA that differs from naturally-occurring RNA by the addition, deletion, substitution and/or alteration of one or more nucleotides. Such alterations can include addition of non-nucleotide material, such as to the end of the molecule or to one or more internal nucleotides of the RNAi, including modifications that make the RNAi resistant to nuclease digestion.

RNAi may be administered in free (naked) form or by the use of delivery systems that enhance stability and/or targeting, e.g., liposomes, or incorporated into other vehicles, such as hydrogels, cyclodextrins, biodegradable nanocapsules, bioadhesive microspheres, or proteinaceous vectors (WO 00/53722), or in combination with a cationic peptide (US 2007275923). They may also be administered in the form of their precursors or encoding DNAs.

Antisense nucleic acid can also be used to down-regulate the expression of the calcineurin. The antisense nucleic acid can be complementary to all or part of a sense nucleic acid encoding a calcineurin subunit e.g., complementary to the coding strand of a double-stranded cDNA molecule or complementary to an mRNA sequence, and it thought to interfere with the translation of the target mRNA; Preferably, the antisense nucleic acid is a RNA molecule complementary to a target mRNA encoding a calcineurin subunit.

An antisense nucleic acid can be, for example, about 5, 10, 15, 20, 25, 30, 35, 40, 45 or 50 nucleotides in length. Particularly, antisense RNA molecules are usually 18-50 nucleotides in length.

An antisense nucleic acid can be constructed using chemical synthesis and enzymatic ligation reactions using procedures known in the art. Particularly, antisense RNA can be chemically synthesized, produced by in vitro transcription from linear (e.g. PCR products) or circular templates (e.g., viral or non-viral vectors), or produced by in vivo transcription from viral or non-viral vectors.

Antisense nucleic acid may be modified to have enhanced stability, nuclease resistance, target specificity and improved pharmacological properties. For example, antisense nucleic acid may include modified nucleotides designed to increase the physical stability of the duplex formed between the antisense and sense nucleic acids, e.g., phosphorothioate derivatives and acridine substituted nucleotides.

Ribozyme molecules can also be used to block the expression of a calcineurin subunit. Ribozymes are catalytic RNA molecules with ribonuclease activity which are capable of cleaving a single-stranded nucleic acid, such as an mRNA, to which they have a complementary region. Thus, ribozymes can be used to catalytically cleave mRNA transcripts to thereby inhibit translation of the protein encoded by the mRNA. Ribozyme molecules specific for a calcineurin subunit can be designed, produced, and administered by methods commonly known to the art (see e.g., Fanning and Symonds, 2006, reviewing therapeutic use of hammerhead ribozymes and small hairpin RNA).

In a particular embodiment, the interfering nucleic acid molecule is expressed by a vector, preferably a viral vector comprising a contruct allowing the expression of interfering nucleic acid molecule. For instance, the viral vector can be an adenovirus, an adeno-associated virus, a lentivirus or a herpes simplex virus.

Calcineurin inhibitor can also be a dominant-interfering form of calcineurin, in particular of CaNA and/or of CaNB. The dominant-interfering form of calcineurin can be for example CaNA-D130N. Other examples of dominant-interfering form of calcineurin are

CaNA-H101Q (Wang et al, 1999, Science 284, 339-343), CaNA-H160Q and CaNA-H290Q (Shibasaki et al, 1996, Nature, 6589, 370-373; Nishimura and Tanaka, 2001, J. Biol. Chem., 276, 19921-19928), H160Q (Zhu et al, 2000n J. Biol. Chem., 275, 15239-15245), D148-152 (Yamashita, 2000, J. Exp. Med., 191, 1869-1880) and Dnter-DcaM (muramatsu and Kincaid, 1996, BBRC, 218, 466-472; Musaro et al, 1999, Nature, 6744, 581-585). Therefore, the dominant-interfering form of calcineurin is expressed by a vector, preferably a viral vector comprising a contruct allowing its expression. For instance, the viral vector can be an adenovirus, an adeno-associated virus, a lentivirus or a herpes simplex virus.

Alternatively, the activity of calcineurin can be inhibited by a drug that inhibits the interaction between the calcineurin subunits, in particular the interaction between subunits A and B. The activity of calcineurin can be inhibited by a drug that inhibits the interaction between the calcineurin and calmodulin. Calcineurin inhibition can also be obtained by activation of endogenous inhibitors of calcineurin, including cabin1, calcipressins and AKAP79.

Other drugs inhibiting the calcineurin can be identified by screening methods already disclosed in the art. As illustration, the U.S. Pat. Nos. 6,875,581 and 6,338,946 describe screening methods useful for identifying modulators of calcineurin activity.

In another embodiment, the compound of the invention increases the phosphorylation of htt. Preferably, the compound increases the phosphorylation of htt at position S421. This compound may increase the activity of a kinase phosphorylating htt. In a particular embodiment, the compound increases the activity of a kinase selected from the group of protein kinase A, Polo kinase 1, cyclin-dependent kinase 5 (Cdk5), kinase Akt, AuroraA and AuroraB and/or SGK. Preferably, the compound increases the activity of a kinase selected from the group of protein kinase A, Polo kinase 1, cyclin-dependent kinase 5 (Cdk5), cyclin-dependent kinase 1 (Cdk1), cyclin-dependent kinase 2 (Cdk2), AuroraA and AuroraB and/or SGK. The compound increasing the phosphorylation of htt may be a nucleic acid encoding a protein kinase involved in the phosphorylation of htt. This compound may also increase the cellular level of a kinase phosphorylating htt by increasing the expression level of the endogenous kinase.

In a further embodiment, the compound of the invention increases the cellular level of phosphorylated htt by increasing the cellular level of wild-type htt. Said compound may be selected from the group consisting of huntingtin protein and a biologically active fragment thereof, huntingtin protein comprising the mutation S241D and a biologically active fragment thereof, and a nucleic acid encoding thereof.

In an embodiment, the compound of the invention is wild-type htt.

In another embodiment, the compound of the invention is a biologically active fragment of htt protein. In a particular embodiment, the biologically active fragment of htt is a N-terminal fragment of the wild-type htt protein comprising at least 500 residues. This fragment may be obtained, for example, by proteolysis of htt by caspase-3 or caspase-6. The term “biologically active fragment of huntingtin protein”, as used herein, refers to a fragment of htt which is able to maintain a normal cell motility. As the inventors have demonstrated that the knock-down of endogenous htt increases cell motility, the term “biologically active fragment of huntingtin protein” refers to a fragment of htt which is able to rescue the increased motility induced by this knock-down. This capacity may be assessed in a cell co-transfected with a RNA construct blocking the expression of htt, e.g. a shRNA htt, and with a nucleic acid encoding the htt fragment to be tested. The motility of cell is then measured and compared to the motility of a control cell transfected only with a RNA construct blocking the expression of endogenous htt. A reduced cell motility compared to the motility of the control cell indicates that the htt fragment encoded by the nucleic acid is biologically active and can be used in the present invention. This test is further detailed in the experimental section.

In a preferred embodiment, the compound of the invention is huntingtin protein comprising the mutation S421D. This protein has been previously described in Humbert et al. (Humbert et al., 2002). The mutation S421D mimics constitutive phosphorylation of htt. In a particular embodiment, the compound of the invention is a biologically active fragment of huntingtin protein comprising the mutation S421D, preferably a N-terminal fragment of said htt protein comprising at least 500 residues. The activity of said fragment may be assessed as described above.

Polypeptides, including htt proteins and their fragments, used in the present invention may be recombinant, purified or isolated polypeptides. The terms “recombinant polypeptide” is used herein to refer to polypeptides that have been artificially designed. A recombinant polypeptide is usually a polypeptide which has been expressed from a recombinant nucleic acid molecule. The term “purified polypeptide” is used herein to describe a polypeptide which has been separated from other compounds including, but not limited to, lipids, carbohydrates, nucleic acids and other proteins. A polypeptide is substantially pure when at least about 50%, preferably 60 to 75%, more preferably 80 to 95%, and even more preferably 95 to 99% of a sample exhibits a single polypeptide sequence. Polypeptide purity or homogeneity may be verified by a number of means well known in the art, such as polyacrylamide gel electrophoresis. The term “isolated” requires that the material is removed from its original environment. The term “isolated polypeptide” refers to a polypeptide that is separated from some or all of the coexisting materials in its natural environment and preferably substantially free from any other contaminating polypeptides which would interfere with its therapeutic use.

In a particular embodiment, the compound increasing the cellular level of wild-type htt is a nucleic acid encoding htt protein or htt protein comprising the mutation. S421D or any biologically active fragment thereof. The nucleic acid may be inserted in a vector allowing its expression in eukaryote cells. The structure and composition of such vectors is well-known by the skilled person. Preferably, the vector is a viral vector such as an adenovirus, an adeno-associated virus, a lentivirus or a herpes simplex virus.

The pharmaceutical composition comprising a compound increasing the cellular level of the phosphorylated form of htt is formulated in accordance with standard pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York) known by a person skilled in the art.

Possible pharmaceutical compositions include those suitable for oral, rectal, topical (including transdermal, buccal and sublingual), intratumoral or parenteral (including subcutaneous, intramuscular, intravenous and intradermal) administration. For these formulations, conventional excipient can be used according to techniques well known by those skilled in the art.

The compositions for parenteral administration are generally physiologically compatible sterile solutions or suspensions which can optionally be prepared immediately before use from solid or lyophilized form. Adjuvants such as a local anesthetic, preservative and buffering agents can be dissolved in the vehicle and a surfactant or wetting agent can be included in the composition to facilitate uniform distribution of the active ingredient.

For oral administration, the composition can be formulated into conventional oral dosage forms such as tablets, capsules, powders, granules and liquid preparations such as syrups, elixirs, and concentrated drops. Non toxic solid carriers or diluents may be used which include, for example, pharmaceutical grades of mannitol, lactose, starch, magnesium stearate, sodium saccharine, talcum, cellulose, glucose, sucrose, magnesium, carbonate, and the like. For compressed tablets, binders, which are agents which impart cohesive qualities to powdered materials are also necessary. For example, starch, gelatine, sugars such as lactose or dextrose, and natural or synthetic gums can be used as binders. Disintegrants are also necessary in the tablets to facilitate break-up of the tablet. Disintegrants include starches, clays, celluloses, algins, gums and crosslinked polymers. Moreover, lubricants and glidants are also included in the tablets to prevent adhesion to the tablet material to surfaces in the manufacturing process and to improve the flow characteristics of the powder material during manufacture. Colloidal silicon dioxide is most commonly used as a glidant and compounds such as talc or stearic acids are most commonly used as lubricants.

For transdermal administration, the composition can be formulated into ointment, cream or gel form and appropriate penetrants or detergents could be used to facilitate permeation, such as dimethyl sulfoxide, dimethyl acetamide and dimethylformamide.

For transmucosal administration, nasal sprays, rectal or vaginal suppositories can be used. The active compound can be incorporated into any of the known suppository bases by methods known in the art. Examples of such bases include cocoa butter, polyethylene glycols (carbowaxes), polyethylene sorbitan monostearate, and mixtures of these with other compatible materials to modify the melting point or dissolution rate.

Pharmaceutical compositions according to the invention may be formulated to release the active drug substantially immediately upon administration or at any predetermined time or time period after administration.

Pharmaceutical compositions according to the invention can comprise one or more compounds increasing the cellular level of the phosphorylated form of htt, associated with pharmaceutically acceptable excipients and/or carriers. These excipients and/or carriers are chosen according to the form of administration as described above.

In a particular embodiment, the pharmaceutical composition comprises one or more compounds increasing the cellular level of the phosphorylated form of htt, selected from the group consisting of a compound inhibiting the dephosphorylation of htt, a compound increasing the phosphorylation of htt and a compound increasing the cellular level of htt.

In another particular embodiment, the pharmaceutical composition comprises one or more compounds increasing the cellular level of the phosphorylated form of htt, selected from the group consisting of a compound inhibiting the dephosphorylation of htt and a compound increasing the cellular level of htt. Preferably, the pharmaceutical composition comprises a compound inhibiting the dephosphorylation of htt and a compound increasing the cellular level of htt. In a particular embodiment, the pharmaceutical composition comprises a calcineurin inhibitor and a compound selected from the group consisting of huntingtin protein and a biologically active fragment thereof, huntingtin protein comprising the mutation S241D and a biologically active fragment thereof, and a nucleic acid encoding thereof.

Other active molecules can also be associated with the compound of the invention such as other molecules used for the treatment of cancer, in particular antitumoral drugs such as tamoxifen, aromatase inhibitors, trastuzumab, GnRH-analogues, gemcitabine, docetaxel, paclitaxel, mitomycin, cisplatin, carboplatin, oxaliplatin, doxorubicin, daunorubicin, docetaxel, cyclophosphamide, epirubicin, fluorouracil, methotrexate, mitozantrone, vinblastine, vincristine, vinorelbine, bleomycin, estramustine phosphate or etoposide phosphate.

The amount of compound increasing the cellular level of the phosphorylated form of htt, to be administered has to be determined by standard procedure well known by those of ordinary skill in the art. Physiological data of the patient (e.g. age, size, and weight), the routes of administration and the type of cancer to be treated have to be taken into account to determine the appropriate dosage.

The compound of the invention may be administered as a single dose or in multiple doses.

In an embodiment, the compound of the invention is a calcineurin inhibitor selected from the group consisting of FK506, cyclosporin A, FK520, L685,818, FK523, 15-0-DeMe-FK-520, Lie120, fenvalerate, resmethrin, cypermethrin, deltamethrin and an analogue thereof and the dose can be, for instance, from 0.001 mg/kg/day to 10 mg/kg/day, preferably between 0.01 and 10 mg/kg/day by oral administration and between 0.001 and 1 mg/kg/day by intravenous injection, preferably between 0.01 and 0.5 mg/kg/day. The unitary dose may be, for instance, from 0.02 mg to 700 mg, preferably from 0.2 mg to 700 mg, for oral administration and from 0.02 mg to 70 mg, preferably from 0.2 mg to 35 mg, for intravenous injection. In a particular embodiment, the calcineurin inhibitor is FK506 and the administered dose of FK506 can be adapted in order to obtain a blood FK506 level comprised between 5 and 40 ng/ml, preferably between 15 and 20 ng/ml.

In another embodiment, the compound of the invention is a calcineurin inhibitor is a nucleic acid molecule interfering specifically with calcineurin expression, such as a RNAi, an antisense nucleic acid or a ribozyme, and the dose can be, for instance, from 1 μg/kg/day to 10 mg/kg/day. The unitary dose may be, for instance, from 70 μg to 700 mg.

In another embodiment, the compound of the invention is a calcineurin inhibitor is a dominant-interfering form of calcineurin and the dose can be, for instance, from 1 μg/kg/day to 10 mg/kg/day. The unitary dose may be, for instance, from 70 μg to 700 mg.

If the compound of the invention is selected from the group consisting of huntingtin protein and a biologically active fragment thereof, huntingtin protein comprising the mutation S241D and a biologically active fragment thereof, and a nucleic acid encoding thereof, the dose can be, for instance, from 1 μg/kg/day to 10 mg/kg/day. The unitary dose may be, for instance, from 70 μg to 700 mg.

In a further embodiment, the compound of the invention is a compound increasing the activity of the kinase Akt, protein kinase A, Polo kinase 1, AuroraA and AuroraB and/or SGK, and the dose can be, for instance, from 1 μg/kg/day to 10 mg/kg/day. The unitary dose may be, for instance, from 70 μg to 700 mg.

The compound of the invention can be used in combination with other active ingredients, in particular with other molecules used for the treatment of cancer. In this case, the compound of the invention and the other molecules can be administered simultaneously or consecutively.

The treatment of cancer with pharmaceutical composition according to the invention can be associated with other therapy such as surgery, radiation therapy or other chemotherapy.

The subject to treat is any mammal, preferably a human being. In a particular embodiment, the subject is a human being not affected by HD.

The present invention also concerns a method for preventing metastasis occurrence and/or cancer invasion in a subject affected with a cancer, the method comprising administering a therapeutically effective amount of a compound increasing the cellular level of the phosphorylated form of huntingtin.

In an embodiment, the compound increasing the cellular level of the phosphorylated form of huntingtin is used to prevent metastasis occurrence in a subject affected with a cancer.

In another embodiment, the compound increasing the cellular level of the phosphorylated form of huntingtin is used to prevent cancer invasion in a subject affected with a cancer.

The term “to prevent metastasis occurrence and/or cancer invasion”, as used herein, refers to delay, minimize or inhibit the spread of cancer through metastasis and/or invasion of surrounding tissues.

The present invention also concerns the use of a compound increasing the cellular level of the phosphorylated form of htt for the manufacture of a medicament for treating cancer.

The present invention further concerns the use of a compound increasing the cellular level of the phosphorylated form of htt for the manufacture of a medicament for preventing metastasis occurrence and/or cancer invasion in a subject affected with a cancer.

The present invention further concerns a method for treating a cancer in a subject, said method comprising the administration of a therapeutically effective amount of a compound increasing the cellular level of the phosphorylated form of huntingtin to said subject. Such compound may inhibit the dephosphorylation of htt, increase the phosphorylation of htt or increase the cellular level of htt, as described above.

In an embodiment, the phosphorylated htt may be phosphorylated at one or several positions selected from the group consisting of S421, S535, S1181, S1201, S2076, S2653 and S2657. Preferably, htt is phosphorylated at position S421.

In a preferred embodiment, the cancer to be treated is an invasive cancer and/or a cancer capable of metastasis. The cancer may be selected from the group consisting of leukemia, lymphoma, melanoma, lung cancer, bowel cancer, colon cancer, rectal cancer, colorectal cancer, brain cancer, liver cancer, pancreatic cancer, breast cancer, prostate cancer, testicular cancer and retinoblastoma. Preferably, the cancer is a breast cancer or a prostate cancer, more preferably a breast cancer.

In a particular embodiment, the method further comprises the step of assessing the cellular level of phosphorylated form of htt in a cancer sample from the subject. The cellular level of phosphorylated form of htt may be determined by any method known by the skilled person, such as method as described below.

The present invention further concerns a method for preventing metastasis occurence and/or cancer invasion in a subject affected with a cancer, said method comprising the administration of a therapeutically effective amount of a compound increasing the cellular level of the phosphorylated form of huntingtin to said subject.

The invention also concerns a method for decreasing the aggressivity of a cancer in a subject, said method comprising the administration of a therapeutically effective amount of a compound increasing the cellular level of the phosphorylated form of huntingtin to said subject.

The inventors herein demonstrate that htt is phosphorylated in normal tissues and that this phosphorylation is lost in cancer cells, in a greater extent in invasive cancer cells. The phosphorylation state of htt thus constitutes a marker for diagnosis of multiple types of cancer, in particular breast cancer.

Accordingly, in another aspect, the present invention concerns a method for diagnosing or detecting a cancer in a subject, wherein the method comprises the step of determining the cellular level of phosphorylated form of htt in a sample from said subject, a low cellular level of phosphorylated htt indicating that said subject suffers from a cancer.

In an embodiment, the method further comprises the step of providing a sample from the subject. Preferably, this sample is suspected to contain tumoral cells.

The cellular level of phosphorylated form of htt may be determined by any method known by the skilled person. For instance, the cellular level of phosphorylated form of htt may be determined by immunohistochemical staining of the sample with an antibody specific to the phosphorylated form of htt, such as the anti-phospho-huntingtin-S421-763 described in the article of Humbert et al. (Humbert et al., 2002), the anti-phospho-huntingtin-S1181 and the anti-phospho-huntingtin-S1201 described in the article of Anne et al. (Anne et al 2007). In a particular embodiment, the cellular level of phosphorylated htt is obtained by measuring the staining intensity of cells in the sample.

In an embodiment, the method further comprises the step of comparing the cellular level of phosphorylated htt to a reference cellular level. Preferably, the reference cellular level is the cellular level of phosphorylated htt in a normal sample. More preferably, the reference cellular level is the mean value of the cellular levels of phosphorylated htt in a panel of normal samples. The normal sample is, as described above, a non-tumoral sample, preferably from the same tissue than the sample to be tested. The normal sample may be obtained from the subject to be diagnosed or from another subject, preferably a healthy subject.

In a further embodiment, the method further comprises the step of determining whether the cellular level of phosphorylated htt is low compared to the reference cellular level. In a particular embodiment, the cellular level of phosphorylated htt in the sample to be tested is considered as low if the level is at least 2, 3, 4, 5, 6, 7, 8, 9 or 10-fold lower than the reference cellular level.

In another embodiment, the present method further comprises assessing at least one other marker used to diagnose cancer such as tumor grade, mitotic index, tumor size or expression of proliferation markers such as Ki67, MCM2, CEA, CA19-9, CA125, PSA, β-hCG or CA15-3.

These markers are commonly used for diagnostic purposes and the results obtained with these markers may be combined with the results obtained with the present method in order to confirm the diagnosis.

The present invention also concerns a method for providing information useful for the diagnosis of a cancer in a subject, wherein the method comprises the step of determining the cellular level of phosphorylated form of htt in a sample from said subject, a low cellular level of phosphorylated htt indicating that said subject suffers from a cancer.

In an embodiment, the method further comprises the step of providing a sample from the subject. Preferably, this sample is suspected to contain tumoral cells.

In another embodiment, the method further comprises the step of comparing the cellular level of phosphorylated htt to a reference cellular level. Preferably, the reference cellular level is the cellular level of phosphorylated htt in a normal sample. More preferably, the reference cellular level is the mean value of the cellular levels of phosphorylated htt in a panel of normal samples.

As demonstrated in the experimental section, the inventors show that cells with decreased level of htt or decreased level of phosphorylated htt or with a poly-Q expansion of htt comprising more than 20 glutamine residues exhibit increased cell motility and increased capacity of metastasis and invasion of surrounding tissues. The expression level of htt, the cellular level of phosphorylated htt and the number of glutamine residues on the poly-Q expansion of htt thus constitute new markers for prognosis.

Accordingly, the present invention further concerns a method for predicting, prognosing or monitoring clinical outcome of a subject affected with a cancer, wherein the method comprises the step of determining the expression level of huntingtin in a cancer sample from said subject, a low expression level of huntingtin being indicative of a poor prognosis.

In an embodiment, the method further comprises the step of providing a cancer sample from the subject.

The expression level of htt can be determined from a cancer sample by a variety of techniques. In an embodiment, the expression level of htt is determined by measuring the quantity of htt protein or htt mRNA.

In a particular embodiment, the expression level of htt is determined by measuring the quantity of htt protein. The quantity of htt protein may be measured by any methods known by the skilled person. Usually, these methods comprise contacting the sample with a binding partner capable of selectively interacting with the htt protein present in the sample. The binding partner is generally a polyclonal or monoclonal antibody, preferably monoclonal. Polyclonal and monoclonal antibodies anti-htt are commercially available such as anti-htt HU-4C8 (Euromedex), HU-2E8 (Eurogentec), HU-2C1 (Euromedex), HU-4E6 (Millipore).

The quantity of htt protein may be measured by semi-quantitative Western blots, enzyme-labeled and mediated immunoassays, such as ELISAs, biotin/avidin type assays, radioimmunoassay, immunoelectrophoresis or immunoprecipitation. The protein expression level may be assessed by immunohistochemistry on a tissue section of the cancer sample. Preferably, the quantity of htt protein is measured by immunohistochemistry or semi-quantitative western-blot.

In another embodiment, the expression level of htt is determined by measuring the quantity of htt mRNA. Methods for determining the quantity of mRNA are well known in the art. For example the nucleic acid contained in the sample (e.g., cell or tissue prepared from the patient) is first extracted according to standard methods, for example using lytic enzymes or chemical solutions or extracted by nucleic-acid-binding resins following the manufacturer's instructions. The extracted mRNA is then detected by hybridization (e.g., Northern blot analysis) and/or amplification (e.g., RT-PCR). Preferably quantitative or semi-quantitative RT-PCR is preferred. Real-time quantitative or semi-quantitative RT-PCR is particularly advantageous. Preferably, primer pairs were designed in order to overlap an intron, so as to distinguish cDNA amplification from putative genomic contamination. Suitable primers may be easily designed by the skilled person. Other methods of Amplification include ligase chain reaction (LCR), transcription-mediated amplification (TMA), strand displacement amplification (SDA) and nucleic acid sequence based amplification (NASBA). Preferably, the quantity of htt mRNA is measured by quantitative or semi-quantitative RT-PCR or by real-time quantitative or semi-quantitative RT-PCR.

In an embodiment, the method further comprises the step of comparing the expression level of htt to a reference expression level.

In a particular embodiment, the reference expression level is the expression level of htt in a cancer sample without invasive or metastatic capacity. Preferably, the reference expression level is the mean value of expression levels of phosphorylated htt in a panel of cancer samples without invasive or metastatic capacity. This sample may be obtained from the subject to be diagnosed or from another subject. Preferably, this sample is from the same tissue than the sample to be tested.

In a further embodiment, the method further comprises the step of determining whether the expression level of htt is low compared to the reference expression level. In a particular embodiment, the expression level of htt in the sample is considered as low if, after normalization, the level is at least 2, 3, 4, 5, 6, 7, 8, 9 or 10-fold lower than the reference expression level. Normalization of expression levels may be easily done by any method known by the skilled person.

In another embodiment, the present method further comprises assessing at least one other prognosis marker such as tumor grade, hormone receptor status, mitotic index, tumor size or expression of proliferation markers such as Ki67, MCM2, CEA, CA19-9, CA125, PSA, β-hCG or CA 15-3. These markers are commonly used and the results obtained with these markers may be combined with the results obtained with the present method in order to confirm the prognosis. The use of these markers is well-known by the skilled person.

The present invention also concerns a method for predicting, prognosing or monitoring clinical outcome of a subject affected with a cancer, wherein the method comprises the step of determining the cellular level of phosphorylated huntingtin in a cancer sample from said subject, a low cellular level of phosphorylated huntingtin being indicative of a poor prognosis.

In an embodiment, the method further comprises the step of providing a cancer sample from the subject.

The cellular level of phosphorylated form of htt may be determined by any method known by the skilled person. For instance, the cellular level of phosphorylated form of htt may be determined by immunohistochemical staining such as described above.

In an embodiment, the method further comprises the step of comparing the cellular level of phosphorylated htt to a reference cellular level.

In a particular embodiment, the reference cellular level is the cellular level of phosphorylated htt in a cancer sample without invasive or metastatic capacity. Preferably, the reference cellular level is the mean value of cellular levels of phosphorylated htt in a panel of cancer samples without invasive or metastatic capacity. This sample may be obtained from the subject to be diagnosed or from another subject. Preferably, this sample is from the same tissue than the sample to be tested.

In a further embodiment, the method further comprises the step of determining whether the cellular level of phosphorylated htt is low compared to the reference cellular level. In a particular embodiment, the cellular level of phosphorylated htt in the sample to be tested is considered as low if the level is at least 2, 3, 4, 5, 6, 7, 8, 9 or 10-fold lower than the reference cellular level.

In another embodiment, the present method further comprises assessing at least one other prognosis marker such as tumor grade, hormone receptor status, mitotic index, tumor size or expression of proliferation markers such as Ki67, MCM2, CEA, CA19-9, CA125, PSA, β-hCG or CA15-3.

In another aspect, the present invention concerns a method for predicting, prognosing or monitoring clinical outcome of a subject affected with a cancer, wherein the method comprises the step of determining the number of glutamine residues on the poly-Q expansion of huntingtin in a sample from said subject, a poly-Q expansion comprising more than 20 glutamine residues being indicative of a poor prognosis.

In an embodiment, a poly-Q expansion comprising more than 35 glutamine residues is indicative of a poor prognosis.

In another embodiment, a poly-Q expansion comprising more than 40 glutamine residues is indicative of a poor prognosis.

The number of glutamine residues on the poly-Q expansion of huntingtin may be determined by any method known by the skilled person. For instance, the htt encoding gene or a fragment thereof comprising the first exon may be amplified and sequenced to determine the number of encoded glutamine residues.

The inventors demonstrate herein that polyQ expansion in htt comprising more than 35 glutamine residues is indicative of an increased likelihood to have an earlier onset of cancer. Accordingly, the present invention further concerns a method for determining the likelihood to have an earlier onset of cancer in a subject, wherein the method comprises the step of determining the number of glutamine residues on the poly-Q expansion of huntingtin in a sample from said subject, a poly-Q expansion comprising more than 35 glutamine residues being indicative of an increase likelihood to have an earlier onset of cancer. In particular, a poly-Q expansion comprising more than 35 glutamine residues is indicative of an increased likelihood to have a cancer before 50 years old. It could be recommended to these subjects to perform earlier cancer screening tests than other subjects with a polyQ expansion in htt comprising 35 or less glutamine residues.

The present invention concerns a method for predicting or prognosing the aggressivity of a cancer in a subject affected with a cancer, wherein the method comprises the step of determining the expression level of huntingtin in a cancer sample from said subject, a low expression level of huntingtin being indicative of an aggressive cancer.

The present invention also concerns a method for predicting or prognosing the aggressivity of a cancer in a subject affected with a cancer, wherein the method comprises the step of determining the cellular level of phosphorylated huntingtin in a cancer sample from said subject, a low cellular level of phosphorylated huntingtin being indicative of an aggressive cancer.

The present invention further concerns a method for predicting or prognosing the aggressivity of a cancer in a subject affected with a cancer, wherein the method comprises the step of determining the number of glutamine residues on the poly-Q expansion of huntingtin in a sample from said subject, a poly-Q expansion comprising more than 20 glutamine residues being indicative of an aggressive cancer. Preferably, a poly-Q expansion comprising more than 35 glutamine residues is indicative of an aggressive cancer. More preferably, a poly-Q expansion comprising more than 40 glutamine residues is indicative of an aggressive cancer.

In a further aspect, the present invention concerns a method for selecting a subject affected with a cancer for an antitumoral therapy, preferably an adjuvant chemotherapy and/or radiotherapy, or determining whether a subject affected with a cancer is susceptible to benefit from an antitumoral therapy, preferably an adjuvant chemotherapy and/or radiotherapy, wherein the method comprises the step of predicting clinical outcome of said subject by any one of methods of the invention as described above, an indication of a poor prognosis indicating that an antitumoral therapy, preferably an adjuvant chemotherapy and/or radiotherapy, is required.

In an embodiment, the method for selecting a subject affected with a cancer for an antitumoral therapy, preferably an adjuvant chemotherapy and/or radiotherapy, or determining whether a subject affected with a cancer is susceptible to benefit from an antitumoral therapy, preferably an adjuvant chemotherapy and/or radiotherapy, comprises the step of determining the expression level of huntingtin in a cancer sample from said subject as described above, a low expression level of huntingtin indicating that an antitumoral therapy, preferably an adjuvant chemotherapy and/or radiotherapy, is required.

In another embodiment, the method for selecting a subject affected with a cancer for an antitumoral therapy, preferably an adjuvant chemotherapy and/or radiotherapy, or determining whether a subject affected with a cancer is susceptible to benefit from an antitumoral therapy, preferably an adjuvant chemotherapy and/or radiotherapy, comprises the step of determining the cellular level of phosphorylated huntingtin in a cancer sample from said subject as described above, a low cellular level of phosphorylated huntingtin indicating that an antitumoral therapy, preferably an adjuvant chemotherapy and/or radiotherapy, is required.

In a further embodiment, the method for selecting a subject affected with a cancer for an antitumoral therapy, preferably an adjuvant chemotherapy and/or radiotherapy, or determining whether a subject affected with a cancer is susceptible to benefit from an antitumoral therapy, preferably an adjuvant chemotherapy and/or radiotherapy, comprises the step of determining the number of glutamine residues on the poly-Q expansion of huntingtin in a sample from said subject as described above, a poly-Q expansion comprising more than 20 glutamine residues indicating that an antitumoral therapy, preferably an adjuvant chemotherapy and/or radiotherapy, is required. Preferably, a poly-Q expansion comprising more than 35 glutamine residues indicates that an antitumoral therapy, preferably an adjuvant chemotherapy and/or radiotherapy, is required. More preferably, a poly-Q expansion comprising more than 40 glutamine residues indicates that an antitumoral therapy, preferably an adjuvant chemotherapy and/or radiotherapy, is required.

In a particular embodiment, the method further comprises assessing at least one other cancer and prognosis marker such as tumor grade, hormone receptor status, mitotic index, tumor size or expression of proliferation markers such as Ki67, MCM2, CEA, CA19-9, CA125, PSA, (β-hCG or CA15-3. The results obtained with these markers may be used to confirm the result obtained with the method according to the invention and/or to orientate the choice of the adjuvant therapy.

The present invention also concerns a method for selecting, identifying or screening a compound useful for treating a subject having cancer, wherein the method comprises the selection or identification of a compound capable of increasing the expression level and/or the phosphorylation of huntingtin.

In an embodiment, the method comprises:

a) providing a huntingtin protein or a fragment thereof of at least 50 consecutive amino acids and comprising at least one phosphorylated residue selected from the group consisting of S421, S535, S1181, S1201, S2076, S2653 and S2657;

b) providing a compound dephosphorylating at least one phosphorylated residue comprised in htt protein or the fragment thereof provided in step a);

c) contacting a candidate compound with said huntingtin protein or fragment thereof and said dephosphorylating compound; and,

d) selecting the candidate compound that inhibits the dephosphorylation of at least one phosphorylated residue comprised in htt protein or the fragment thereof provided in step a) by dephosphorylating compound.

In a particular embodiment, the method comprises:

a) providing a huntingtin protein or a fragment thereof of at least 50 consecutive amino acids and comprising at least a phosphorylated residue at position S421;

b) providing a calcineurin;

c) contacting a candidate compound with said huntingtin protein or fragment thereof and said calcineurin; and,

d) selecting the candidate compound that inhibits the dephosphorylation of the phosphorylated residue of huntingtin at position S421 by calcineurin.

In another embodiment, the method comprises:

a) contacting a candidate compound with a cell expressing a huntingtin protein and comprising a kinase which phosphorylates huntingtin at a position selected from the group consisting of S421, S535, S1181, S1201, S2076, S2653 and S2657, and a compound dephosphorylating the phosphorylated residue at selected position;

b) assessing the amount of huntingtin phosphorylated and/or the amount of huntingtin which is not phosphorylated; and,

c) selecting the candidate compound that increases the phosphorylation of huntingtin at selected position in comparison with a control cell which has not been contacted with the candidate compound.

In a further embodiment, the method comprises:

a) contacting a candidate compound with a cell expressing a huntingtin protein and comprising a kinase which phosphorylates huntingtin at position S421 and a calcineurin;

b) assessing the amount of huntingtin phosphorylated and/or the amount of huntingtin which is not phosphorylated; and,

c) selecting the candidate compound that increases the phosphorylation of huntingtin at position S421 in comparison with a control cell which has not been contacted with the candidate compound.

The phosphorylation of htt may be detected with an anti-phosphorylated htt antibody such as described above.

In a further embodiment, the method comprises:

a) contacting a candidate compound with a cell expressing a huntingtin protein;

b) assessing the amount of huntingtin expressed in said cell; and

c) selecting the candidate compound that increases the expression of huntingtin in comparison with a control cell which has not been contacted with the candidate compound.

In a last aspect, the present invention concerns a kit:

(a) for diagnosing or detecting a cancer in a subject; and/or

(b) for predicting, prognosing or monitoring clinical outcome of a subject affected with a cancer; and/or

(c) for selecting a subject affected with a cancer for an adjuvant therapy and/or radiotherapy or determining whether a subject affected with a cancer is susceptible to benefit from an adjuvant chemotherapy and/or radiotherapy; and/or

wherein the kit comprises:

(i) at least one antibody specific to htt and, optionally, means for detecting the formation of the complex between htt and said at least one antibody; and/or

(ii) at least one antibody specific to phosphorylated htt and, optionally, means for detecting the formation of the complex between htt and said at least one antibody; and/or

(iii) at least one probe specific to the protein htt and, optionally, means for detecting the hybridization of said at least one probe on htt protein; and/or

(iv) at least one nucleic acid primer pair specific to htt gene or mRNA and, optionally, means for amplifying and/or detecting said gene or said mRNA; and,

(v) optionally, a leaflet providing guidelines to use such a kit.

All references cited in this specification are incorporated by reference.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.”

The reference in this specification to any prior publication (or information derived from it), or to any matter which is known, is not, and should not be taken as an acknowledgement or admission or any form of suggestion that that prior publication (or information derived from it) or known matter forms part of the common general knowledge in the field of endeavour to which this specification relates.

Further aspects and advantages of the present invention will be described in the following examples, which should be regarded as illustrative and not limiting.

### EXAMPLES

### Example 1

### HTT and Breast Cancer

Materials and Methods

Antibodies—The antibodies used herein are as follows: monoclonal anti-Htt 4c8 (Euromedex); rabbit anti-Htt 737 (Anne et al; 2007); rabbit anti-phospho-S421-human Htt 763 (Humbert et al, 2002); monoclonal β-actin (Sigma); monoclonal ZO-1 (BD Transduction Laboratories); rabbit anti-firefly luciferase (Abeam); and monoclonal anti-GM-130 (BD Transduction Laboratories). Secondary antibodies for immunofluorescence studies were Alexa 488 (anti-mouse and anti-rabbit), 555 (anti-mouse and anti-rabbit), and Cy5 anti-mouse from Invitrogen. Secondary antibodies for western blots were HRP-conjugated anti-mouse and anti-rabbit from Invitrogen.

Cells—4t1-luc and 67NR cells were maintained in DMEM (Gibco) with 10% fetal calf serum, 1×MEM non-essential amino acids (Gibco), 100 units/mL penicillin/streptomycin (Gibco), 250 ng/mL Fungizone (Gibco), and 400 μg/ml Hygromycin B (Invitrogen). MCF7, CAMA-1 and HEK293 cells were maintained in DMEM with 10% fetal calf serum, 100 units/mL penicillin/streptomycin (Gibco), and 0.1% Fungizone (Gibco). COS-7 cells were maintained in DMEM with 10% fetal calf serum, 100 units/mL penicillin/streptomycin (Gibco), 1% glutamine, and 250 ng/mL Fungizone (Gibco). 2A1 cells were maintained in DMEM with 10% bovine calf serum, 100 units/mL penicillin/streptomycin (Gibco), 1% glutamine, 250 ng/mL Fungizone (Gibco), and 400 μg/ml geneticin (Gibco). SH-SY5Y cells were maintained in RPMI with 10% BCS and 1% glutamine.

2A1 cells were grown at 33° C. and 5% CO₂. All other cells were kept at 37° C. and 5% CO₂.

Plasmids, siRNA and transfections—The hairpin region of shHtt recognizes a region within exons 8-9 (AGCTTTGATGGATTCTAATCTCTTGAAATTAGAATCCATCAAAG CT, SEQ ID NO: 4). Within the same plasmid is a separate open reading frame coding for GFP behind the PGK promoter. The shLuc construct is identical apart from its hairpin recognizing a sequence within the firefly luciferase gene rather than a sequence within htt. The 17QHtt480 plasmids are described in Saudou et al., 1998. mCherry is expressed in the pCDNA 3.2 plasmid. The pARIS-Htt plasmids used herein are variations of a synthetic full-length htt gene with multiple tags in the pCDNA 3.2 plasmid. This plasmid is rendered insensitive to multiple siRNA and shRNA sites, including those used herein, by point mutations in the DNA which do not affect the amino acid sequence. The S421A and S421D mutations were introduced by simple site-directed mutagenesis using the Invitrogen Gateway System.

The siRNA against mouse Htt used herein, previously described in Zala et al., 2008, is complementary to the coding sequence 361-380 of mouse Htt. The control scRNA is a scrambled version of the nucleotides in the siRNA verified to not have complementarity to any other sites in the genome.

Transfections were carried out as follows. For random migration assays in 2A1, cells were lipofected (LIPOFECTAMINE 2000, Invitrogen) or electroporated 2-3 days before assays with either shHTT or shLuc and pCDNA3.2-cherry, or CTFLHtt-WT, -S421A, or -S421D. MCF-7 cells were lipofected 48 hours before scratch assays and before lysis for immunoprecipitation experiments. COS-7 cells were electroporated 24-48 hours before fixation for leading edge quantification studies. HEK293 cells were transfected by calcium phosphate precipitation. siRNA treatment of 4t1 cells was carried out using lipofectamine 2000.

Infection by lentiviral vectors—Using the Gateway subcloning system from Invitrogen, CTFLhtt constructs were cloned into a lentiviral vector. This vector was then transfected into HEK 293T cells. Cells were lysed, and lentiviral particles were collected. Titer was calculated by ELISA assay. 4t1-luc cells in 24-well plates were then infected with 1.125 ug of virus. The media was changed 24 hours later.

Generation of stable cell lines—Expression of CTFLhtt, as observed by fluorescence microscopy, revealed a high but incomplete lentiviral infection rate of 4t1-luc cells, so fluorescent activated cell sorting was carried out to purify the cell population. Subsequent immunocytochemical and immunoblotting studies suggested that the rate of CTFLhtt expressing cells was close to 100% and that the levels of expression between the different cell lines were similar.

Immunohistochemistry—Paraffin imbedded biopsies from breast cancer patients and healthy subjects were obtained from the Curie Institute Hospital pathology department. All samples were de-paraffinized and then de-masked in Dako EDTA pH 9 antigen retrieval solution at 90° C. for 30 minutes. Samples were then probed using a Vectastain ABC kit from Vector laboratories. The 4c8 anti-Htt antibody was used at a concentration of 1:300, and the phospho-Htt 763 antibody was used at a concentration of 1:50. All samples were also counterstained with Hematoxylin to identify nuclei.

Immunofluorescence—To examine the total level of htt in 2a1 cells which had been gene-replaced, cells were fixed with cold methanol and then stained with 737 total htt antibody (1:25). Gene-replaced cells were identifiable by the GFP fluorescence from the shHtt and the cherry fluorescence from the CTFLhtt.

To verify whether 4t1-luc cells from the in vivo metastasis assay were indeed knocked down for htt from the intraperitoneal injections of siHtt, lungs were dissected and then frozen in liquid nitrogen. Samples were then processed for immunofluorescence imaging by cryostat sectioning and adhering to Thermo Scientific Superfrost glass slides. Sections were then fixed in 4% paraformaldehyde and blocked for three days to remove any autofluorescence in 1% BSA+0.1% triton x-100+0.15% glycine. Samples were then probed with antibodies against luciferase (1:500) and htt (4c8 1:400). Imaging was then carried out using a Leica TCS-SP5 confocal microscope with 63× objective lens.

Cell Motility—Random migration experiments were carried out in 2a1 and MCF7 cells. Cells in plastic 6-well plates were transfected, as described above, and then imaged over at least six hours using an inverted fluorescent 2D Leica DM IRB microscope with photometric CoolSNAP fx camera in a chamber with controlled temperature and CO₂ conditions and a moving stage. This system allows for the storage of cell coordinates so that many cells at different positions can be followed during the same time-course. Transfected cells were identified by fluorescence imaging as described above. Dividing cells were excluded from analysis. shLuc- or shHtt-transfected MCF7 cells were induced to move as follows. Cells were trypsinized 16 hours before assay and seeded onto wells of a fibronectin-coated (20 μg/mL) plastic 6-well plate. EGF (Sigma) was then added to 20 ng/mL. Imaging began one hour later using the same protocol as for the 2a1 cells. The cell tracking plug-in of ImageJ software was then used to calculate parameters of cell motility.

Scratch assays were performed in 4t1 and MCF7 cells. Transfected cells were grown 2-3 days until confluence. Then, a 200 μL pipet tip was used to scratch the confluent monolayer. Cells were then imaged for 48 hours using the same microscope configuration as described for random migration assays. ImageJ software was then used to measure the distance between the two columns of cells at specific timepoints. In all cases, the in vitro doubling times for the cells within each condition were confirmed to be the same so treatment with inhibitors of cell division were not necessary.

Matrigel Invasion—Growth factor reduced Matrigel invasion chambers were from BD Biosciences (Reference number 354483). Before addition of cells, chambers were allowed to warm to room temperature for one hour and then re-hydrated in 0.1% serum containing 40 media in a 37° C. incubator at 5% CO₂ for at least three hours. Just before the addition of cells, the chambers were placed into wells containing full serum (10%) containing 411 media. 4t1-luc cells were treated with siRNA or scramble for 24 hours before being added to the chambers. Cells were serum starved in 0.1% serum-containing 4t1 media for at least six hours before being trypsinized and resuspended. 100,000 cells were then resuspended in 500 μL 0.1% serum 4t1 media and added to the top of the chambers. 24 hours later, the matrigel was removed from the chamber, the chamber was rinsed twice with 0.1% serum 4t1 media, and the chambers were fixed in 4% paraformaldehyde for 10 minutes. The chambers were then washed three times in 1×PBS. Next, the chambers were stained with toluidene blue dye for 30 minutes and washed 5 times with water over the course of ten minutes. Then, images were taken of the chambers, still housed in 24-well dishes, using an inverted microscope. The cells were then counted using an ImageJ cell counter plug-in.

In vivo metastasis—The in vivo metastasis assay was done using 4t1-luc cells injected into syngenic BALB/C mice as described previously (Fitamant et al., 2008) Briefly, 100,000 cells resuspended in 150 μL of PBS were tail-vein injected. 4 μg of sc or siRNA was intraperitoneally injected each day for ten days, starting with the day of the cell injections. Each day, beginning at day 4, luciferin was administered to the mice, and bio-luminescence imaging was performed, and the lung signal was quantified. Statistical differences in lung signal intensity were determined by Mann-Whitney U post-hoc analysis. The siRNA used in this study was the mouse siHtt described previously. The scramble control was a previously used sequence containing no homology to known sites of the mouse genome (GAUAGCAAUGACGAAUGCGUA, SEQ ID NO: 5). Survival Assays were performed in compliance with French animal research ethical standards: when mice lost more than 15% of their pre-treatment weight due to the effects of the cancer cells, they were euthanized. The day of euthanasia was recorded as their last day of survival. Control mice treated with sc or siRNA, but never injected with 4t1-luc cells, never showed any loss of weight during the course of the study. Kaplan-Meier survival plots were evaluated by a log-rank analysis for statistical significance.

Results

Htt is Present at High Levels in Normal Breast Epithelia and Cancer Cells

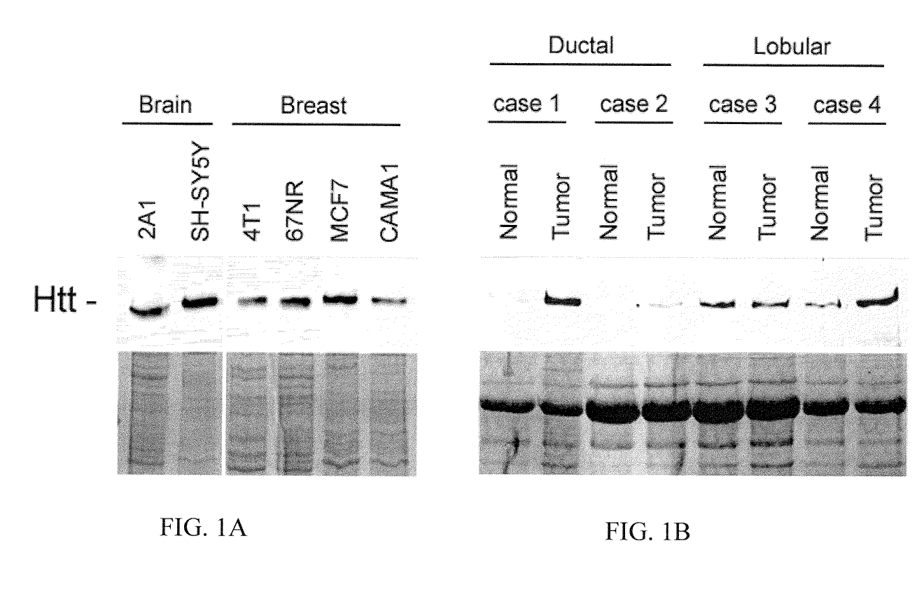

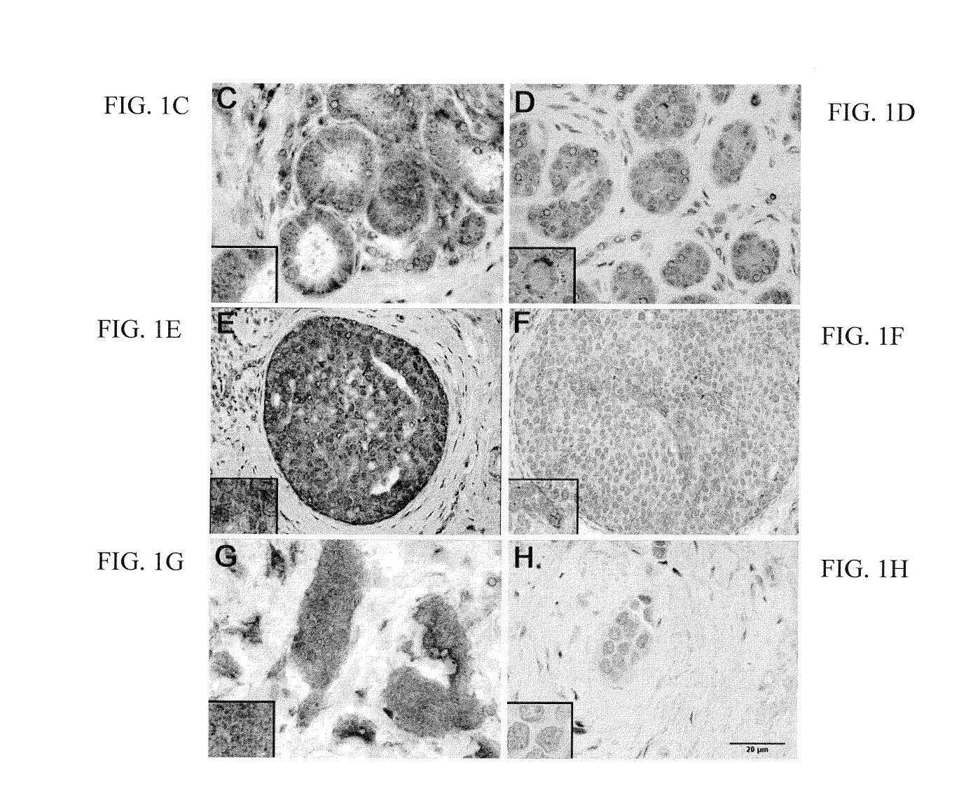

While the expression of Htt is known to be ubiquitous, it is thought to be enriched in the brain. However, the inventors found that htt is present in breast cells lines at a similar level as in neuronal cell lines (FIG. 1A). The finding of a high endogenous level of htt in cultured breast cancer cells prompted an examination of the localization of Htt protein in normal breast tissue. It was discovered that htt was expressed specifically in the epithelial cells lining both mammary glands and ducts and not detectable in stromal cells (FIG. 1C).

The state of htt and its phosphorylation at serine 421 (S421), a modification known to regulate htt toxicity and function, were examined in human breast cancer. To this end, the inventors tested via immunohistochemistry and western blot analysis whether htt was enriched in breast tumor samples. Western blotting revealed that htt was greatly enriched in samples from breast tumor samples as compared to healthy breast tissue from the same individuals (FIG. 1B). However, one can not conclude from these studies whether the enrichment of htt in tumor samples reflects a cellular upregulation of htt protein in the cancer state. In fact, based on immunohistochemical studies, it seems more likely that the enrichment is simply a result of the tumor itself being a proliferation of cells of epithelial origin (compare FIG. 1C to FIG. 1E).

In order to examine the differences in the phosphorylation of S421 in the normal versus cancer conditions, the inventors performed stainings with an antibody (763) specific to the phosphorylated form of htt (pHtt) in normal (FIG. 1D) and cancerous breast tissue (FIGS. 1F and G). They surprisingly found a very specific staining of pHtt to small punctate structures at the apical part of the junctions between breast epithelial cells (FIG. 1D, inset). It was hypothesized that these structures could possibly be adherens junctions or tight junctions.

To investigate whether total htt or pHtt were differentially regulated in the cancer state, immunohistochemical staining of normal tissue was compared to that of cancerous tissue. No difference was found between the intensity of 4C8 staining between normal cells and cancerous ones (FIGS. 1C and E), but a striking difference was observed in pHtt staining when comparing normal to cancerous cells (FIGS. 1D,E,F). The overall trend the inventors noticed was that htt phosphorylation was lost in invasive cancer cells and, to a lesser degree, in primary tumors (FIGS. 1H and F, respectively). The loss of phosphorylation was quantified in invasive cancer cells in breast cancer patient samples that contained both invasive cancer cells and normal glands or ducts as an internal positive control for staining. It was found that, though normal htt staining was present in the invasive cells of all breast cancer samples examined, pHtt staining was lost in 6/9 cases of ductal breast cancer (FIG. 1I) and 10/10 cases of lobular disease (FIG. 1J). A more general correlation was also noticed between the level of differentiation of breast epithelial cells and the presence of htt phosphorylation at residue S421 (data not shown).

Htt Regulates Cell Motility

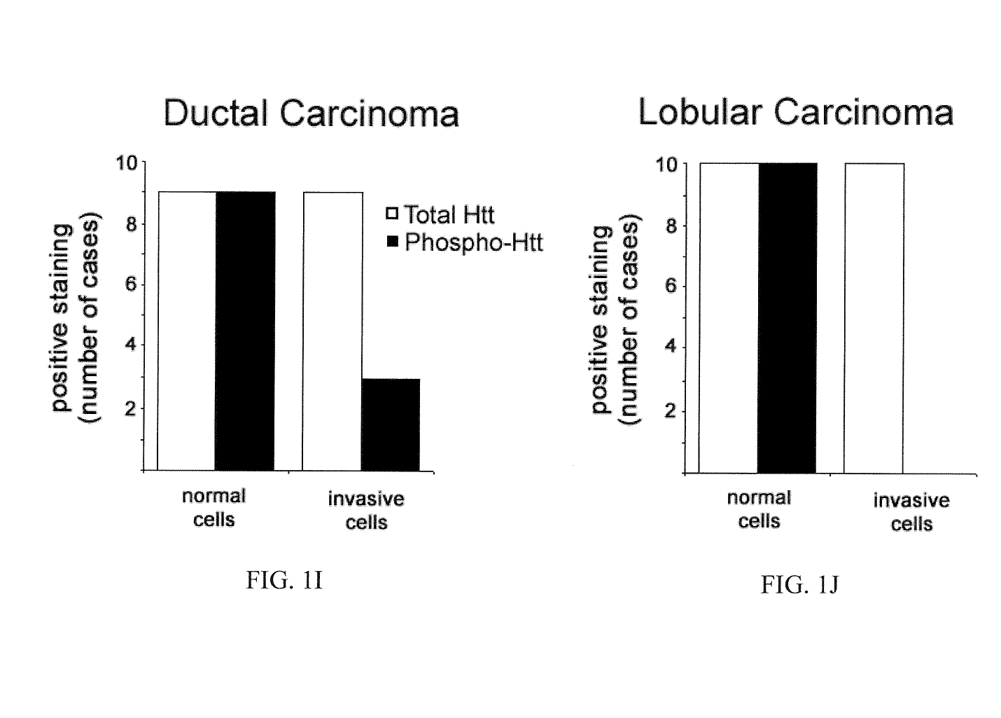

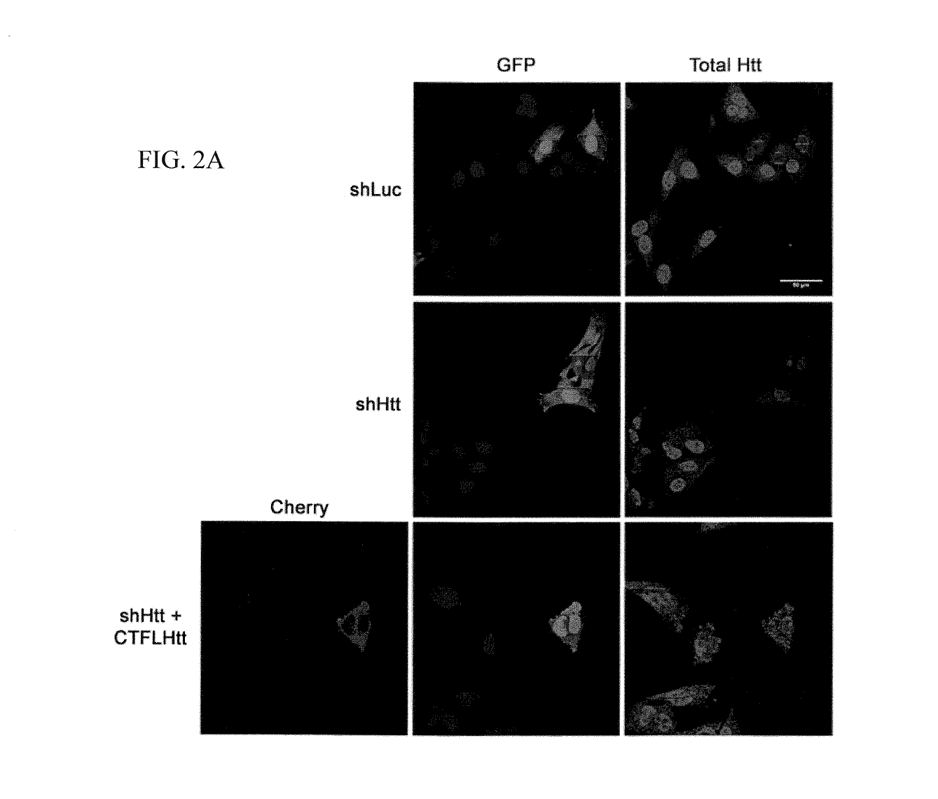

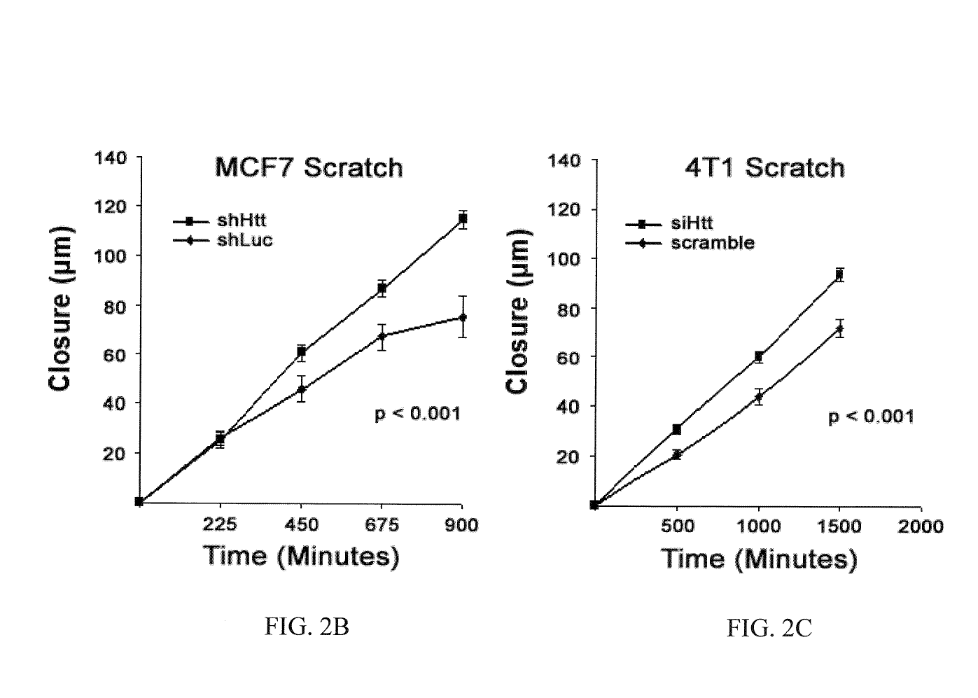

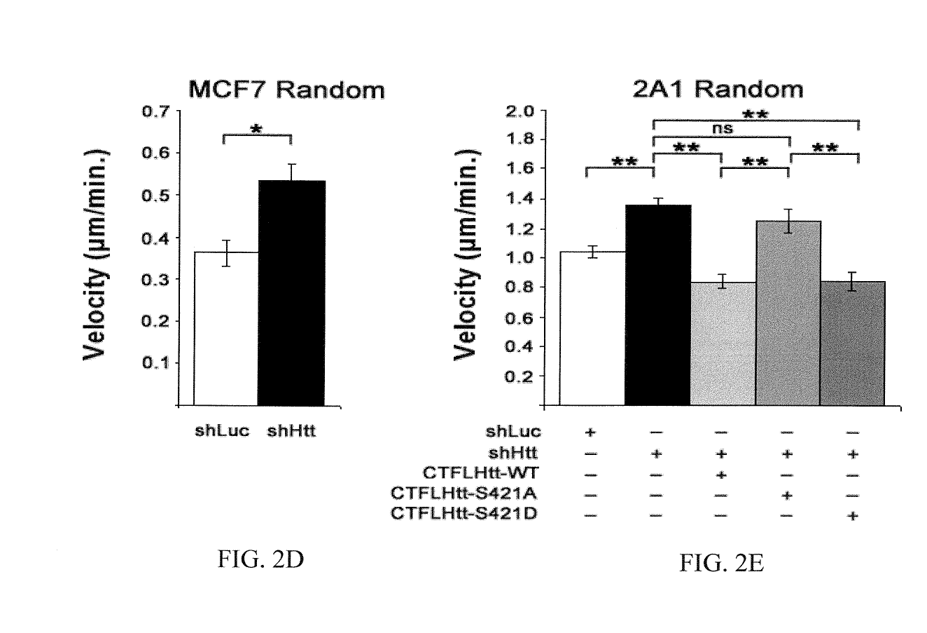

The processes of in vivo invasion and in vitro cell motility share many of the same signaling pathways. In vitro assays were designed to determine whether htt played any role in cell motility. The first approach was a simple scratch assay in MCF7 breast cancer cells. Cells were transfected with either a control small hairpin RNA (shRNA) construct against firefly luciferase (shLuc) or an shRNA against htt (shHtt) to reduce endogenous levels of the protein. Both of these plasmids contain a gene for green fluorescent protein (GFP) behind a separate promoter, allowing the identification of live transfected cells (FIG. 2A, middle column). After transfection, cells were grown to confluence and then scratched with a pipette tip. Closure of the scratch was monitored by live cell imaging and quantified using ImageJ software. It was found that cells in which the endogenous htt was decreased by shHtt showed a faster scratch closure than control cells, suggesting that htt does play a role in cell motility (FIG. 2B). A similar scratch closure assay in 4t1 mouse breast cancer cells transfected by a scramble RNA or an siRNA against htt supported this conclusion (FIG. 2C).

While scratch closure assays are well established to evaluate cell motility in epithelial cells, they likely under-estimate the total effect when cells are transiently transfected with sh- or siRNA due to incomplete transfection. Another limitation is that scratch closure reflects collective cell movement, as opposed to individual cell movement. To address these problems, random cell migration assays were performed in MCF7 cells. To induce individual MCF7 cells to move, plastic culture plates were coated with fibronectin, cells were plated at low density and later epidermal growth factor (EGF) was added. Cells were again transfected with either shLuc or shHtt. Western blot analysis (not shown) and immunofluorescence studies (FIG. 2A, right column) validated the efficacy of shHtt to reduce endogenous htt levels. The results of this assay further supported the previous motility experiments in that cells expressing shHtt moved about 50% faster than the shLuc-expressing control cells.

Though these results were consistent with a role for htt in regulating cell motility, the analysis of the random MCF7 cell migration assay may suffer potential confounds due to the stimulation of cells by fibronectin and EGF. Therefore, a similar experiment was carried out in non-epithelial cells which exhibit individual cell movement without needing to be stimulated by any growth factors. 2a1 cells, a neuronal cell line well established for the study of the htt protein, were chosen. This assay simply required that cells be transfected with shLuc or shHtt and then followed by live cell imaging. As with the MCF7 cells, 2a1 cells expressing shHtt moved about 50% faster than the control shLuc-expressing cells (FIG. 2E, bars shLuc and shHtt)).

Taken together these four separate motility assays demonstrate that decreasing endogenous htt leads cells to move faster.

The inventors' hypothesis based on the in vivo invasion data (FIG. 1) was that when cells in culture lose phosphorylated htt, they would move faster. In order to test this hypothesis, a “gene replacement” experiment was designed. Co-transfections were carried on with shHtt and one of three cherry-tagged, shRNA-resistant full-length Htt plasmids (CTFLhtt): wildtype (WT), a point mutant at serine 421 to an alanine to mimic constitutive dephosphorylation (S421A) or a point mutant at serine 421 to an aspartic acid to mimic constitutive phosphorylation (S421D). In this way, the cellular pool of full-length htt could be manipulated to reflect conditions of high or low phosphorylation of htt at S421. As these cells would express both GFP to identify endogenous-htt depleted cells and cherry to identify cells expressing the point mutants, it was possible to verify that the “gene-replaced” cells expressed levels of total htt similar to untransfected cells (FIG. 1A, lower right panel).

It was assumed that, if phosphorylation of Htt is required for maintaining a basal level of cell motility, then the expression of the WT or S421D constructs would rescue the increased cell motility brought about by decreasing endogenous htt, while the S421A construct would not. The results of the experiment closely agreed with this idea (FIG. 2E). This experiment also rules out the possibility that the increased motility seen in shHtt-expressing cells could be due to off-target effects of the shRNA construct (FIG. 2E, bar shHtt vs. bar shHtt+WT).

In conclusion, some level of htt phosphorylation is required to maintain normal cell motility in vitro. When this level is reduced, for example by shHtt, cells move faster.

Htt Regulates Metastasis In Vivo

Based on the finding in human breast cancer patients that htt phosphorylation is lost in invasive cancer cells and on the finding that the increased cell motility which results from decreasing endogenous htt can be rescued only by phosphorylated htt, an in vivo experiment was designed to examine whether htt could also regulate metastasis. The 4t1-luciferase system was chosen to test this possibility. In this well established experimental paradigm, syngenic mice are tail-vein injected with stably luciferase-expressing breast cancer cells (4t1-luc), and metastasis to lung is monitored by live bioluminescence imaging following systemic injection of luciferin (Fitamant et al., 2008).

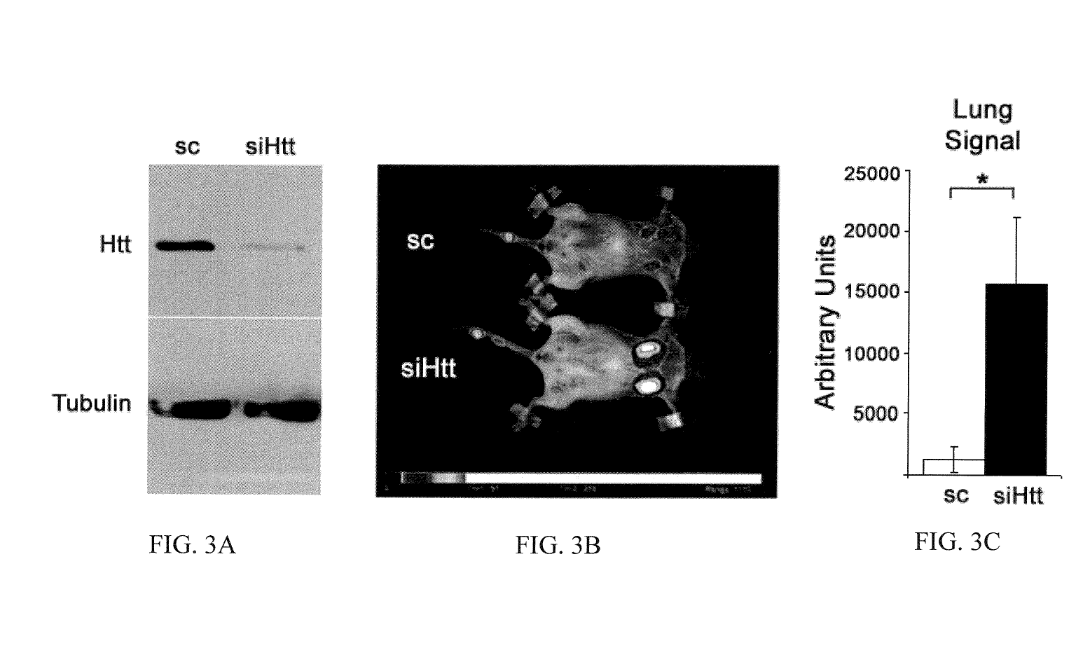

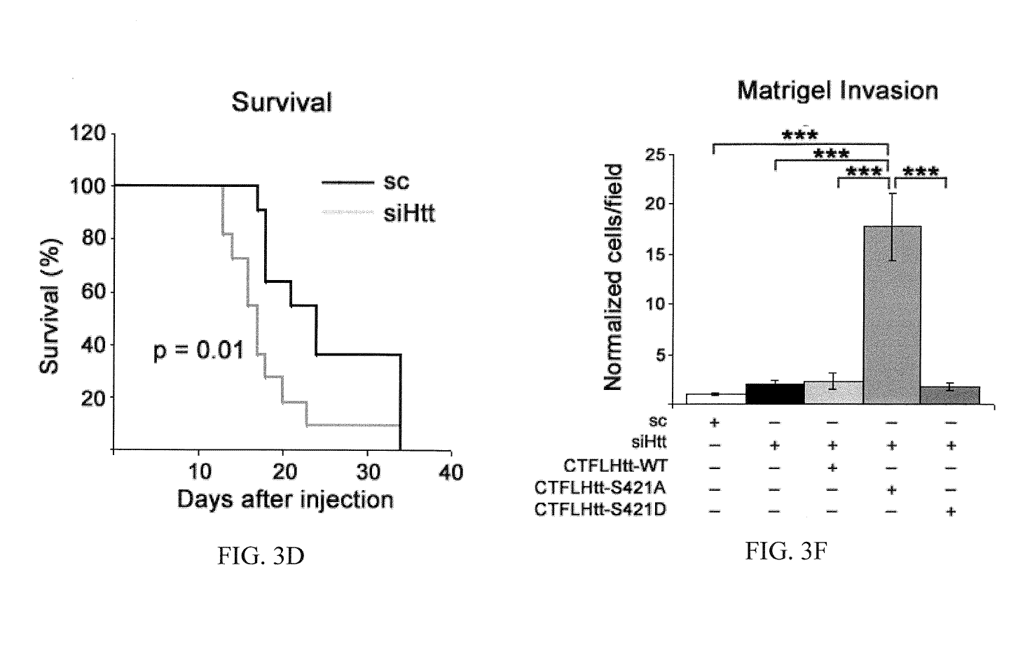

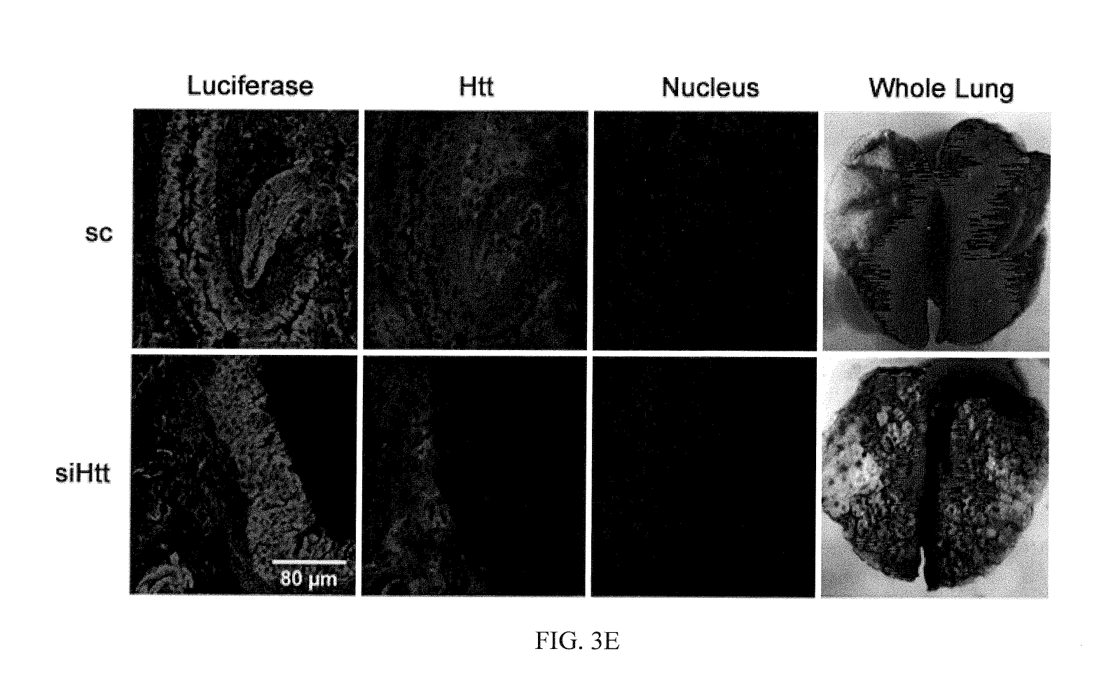

To determine whether htt could modulate metastasis in this system, mice were injected with a scramble control (sc) or an siRNA against htt (siHtt) (FIG. 3A). Seven days after the cancer cell injections, a striking ten-fold increase was found in the luciferase signal in the lungs of siHtt-treated mice as compared to scramble-treated mice (FIGS. 3B,C). Immunofluorescence evaluation of the lungs of siHtt-treated mice revealed that the luciferase-positive 4t1 cells did indeed show a substantially decreased level of htt as compared to control mice (FIG. 3E). Gross evaluation of these dissected lungs indicated a substantially higher level of metastasis in the siHtt-treated mouse. Furthermore, these data correlate well with a statistically significant decrease in the survival of the siHtt-treated mice (FIG. 3D). No siHtt-treated mice not injected with 4t1-luc cells died during the course of the experiment, confirming that this decreased survival was due to an effect of the siHtt on cancer progression in these mice.

Htt Phosphorylation Controls Cancer Cell Invasion

To definitively understand whether changes in htt were causative events in cancer cell invasion or simply silent downstream targets of the true effectors, the inventors tested how manipulating htt might alter cancer cell invasion in matrigel invasion chambers. As in the above motility experiments, the influence of knocking down endogenous htt and replacing it with the different CTFLhtt constructs mutated at S421, was specifically examined. To this end, 4t1-luc cell lines stably expressing the three different CTFLhtt constructs: WT, S421A and S421D, were created. These constructs are resistant to siRNA. Thus, a “gene replacement” strategy was used to examine the effects of htt phosphorylation on invasion.

The results of this experiment demonstrate that when a cell has a higher proportion of unphosphorylated Htt, it is much more capable of invasion. Specifically, 4t1-luc cells which have been gene-replaced with the CTFLS421A mutant show a >15 fold increase in the number of cells which were able to invade through matrigel chambers compared to non-gene replaced 4±1-luc cells (GFP scramble) (FIG. 3F). No such effect was seen in cells which had been gene-replaced for the S421D mutant. This demonstrates that cells require some level of phosphorylated htt to prevent elevated invasive ability. When this relative level is decreased, cells become much more invasive.

### Example 2

### PolyQ-Htt and Breast Cancer

Materials and Methods

Mice—Mice were housed in a 12 hour light/dark cycle and fed a regular diet and water ad libitum. Mice were sacrificed by cervical dislocation. Mammary glands were dissected and spread on glass slides. Mammary glands were fixed overnight in methacarn (60% methanol, 30% chloroform and 10% acetic acid), washed in 100% methanol for 1 hour and left overnight in fresh methanol. For whole mount staining, mammary glands were rehydrated by a bath of 70% ethanol for 15 min, followed by a 5 min bath in H2O and stained overnight with carmine aluminum staining. Stained glands were dehydrated by 15 min baths in ethanol (70%, 95% and 100%), cleared in xylene, and stored in methyl salicylate. Else, paraffin embedded tissue was fixed and stained with hematoxylin and eosin (Taddei et al., 2008). Grafts experiments were performed as previously described for xenografts (Morton and Houghton, 2007) except that 8 mm³ pieces of mouse tumors were grafted in 6 week-old Swiss nude mice.

Random migration assays—Cells in plastic 6-well plates were imaged over 6-8 hours using an inverted fluorescent 2D Leica DM IRB microscope with photometric CoolSNAP fx camera in a chamber with controlled temperature and CO₂ conditions and a moving stage. This system allows for the storage of cell coordinates so that many cells at different positions can be followed during the same time-course. Transfected cells were identified by fluorescence imaging as described above. Dividing cells were excluded from analysis. The cell tracking plug-in of ImageJ software was then used to calculate parameters of cell motility.

Invasion assays—Growth factor reduced Matrigel invasion chambers were from BD Biosciences (Reference number 354483). Before addition of cells, chambers were allowed to warm to room temperature for one hour and then re-hydrated in 0.1% serum containing culture media in a 37° C. incubator at 5% CO₂ for at least three hours. Just before the addition of cells, the chambers were placed into wells containing 10% serum and culture media. Cells were serum starved in 0.1% serum-containing media for at least six hours before being trypsinized and resuspended. 100,000 cells were then resuspended in 500 μl of media containing 0.1% serum and added to the top of the chambers. 24 hours later, the matrigel was removed from the chamber, the chamber was rinsed twice with 0.1% serum media, and the chambers were fixed in 4% paraformaldehyde for 10 minutes. The chambers were then washed three times in 1×PBS, stained with toluidene blue dye for 30 minutes and washed 5 times with water over the course of ten minutes. Then, images were taken of the chambers, still housed in 24-well dishes, using an inverted microscope. The cells were then counted using an ImageJ cell counter plug-in.

Results

Oncogene-Induced Mammary Tumors Develop Faster in HD Mouse Model

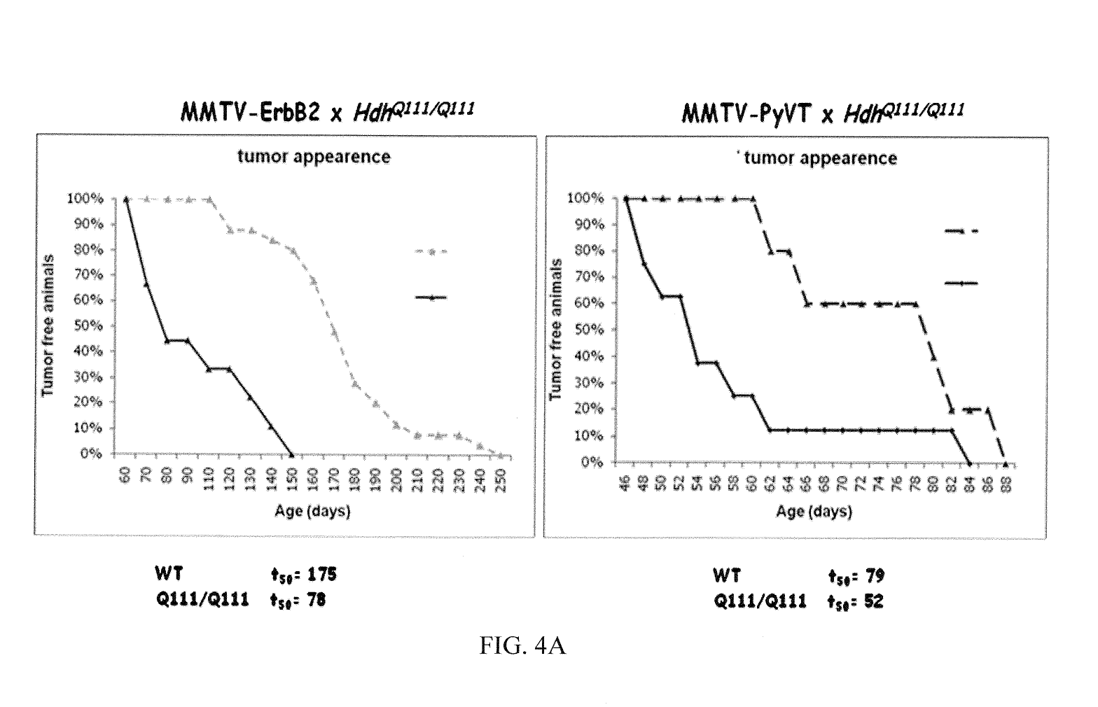

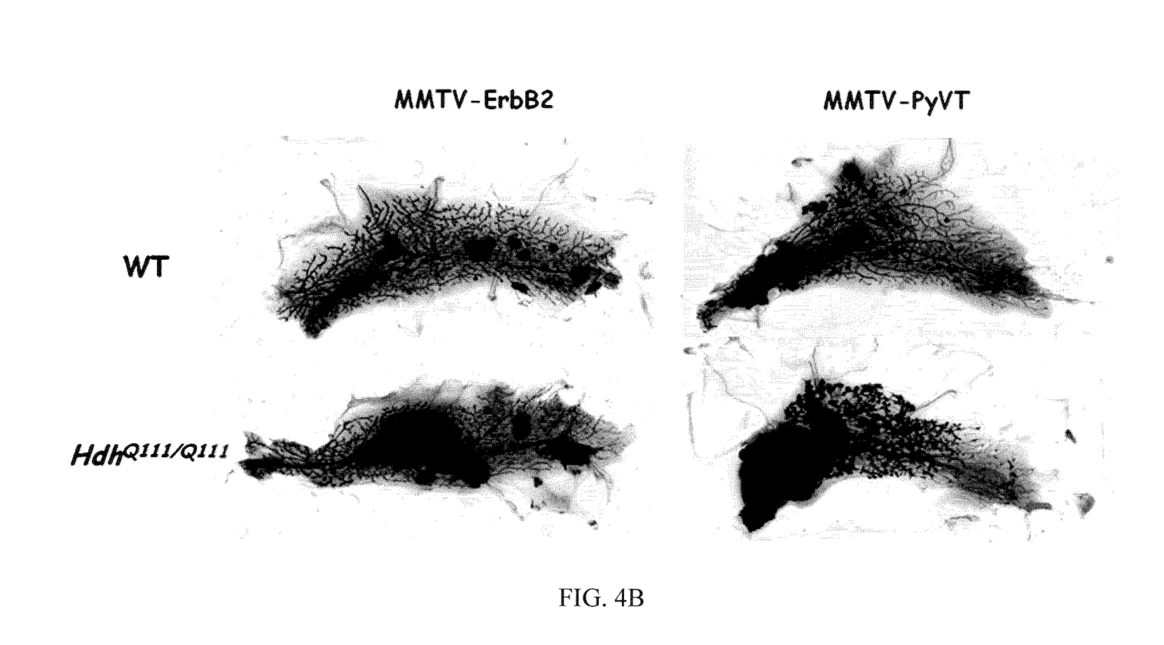

To examine the influence of the presence of polyQ-huntingtin on tumor progression, the inventors derived mice that express activated oncogenes and polyQ-huntingtin. Mice expressing the activated PyVT (Guy et al., 1992) or ErbB2 (Muller et al., 1988) under the control of the MMTV promoter (respectively MMTV-PyVT and MMTV-ErbB2) were crossed with the mouse line carrying a 111 CAG expansion inserted into the endogenous mouse huntingtin gene (Wheeler et al., 2002). Compared with MMTV-PyVT mice expressing wild-type huntingtin (MMTV-PyVT; Hdh^{Q7/Q7}), the appearance of tumors was observed earlier in MMTV-PyVT; Hdh^{Q111/Q111} mice (FIG. 4A). Similar results were obtained using the MMTV-ErbB2 model of mammary cancer in mouse (FIG. 4A). Examination of virgin female mammary whole mount from MMTV-PyVT; Hdh^{Q111/Q111} (FIG. 4B) and MMTV-ErbB2; Hdh^{Q111/Q111} (FIG. 4B) revealed the presence of multiple large mammary adenocarcinomas as compared to the adenocarcinomas induced in the huntingtin wild-type background. These observations suggest that the presence of polyQ huntingtin accelerates mammary carcinogenesis in oncogene-induced mouse models.

PolyQ Tumors do not Grow Faster but Show Increased Activation of Akt

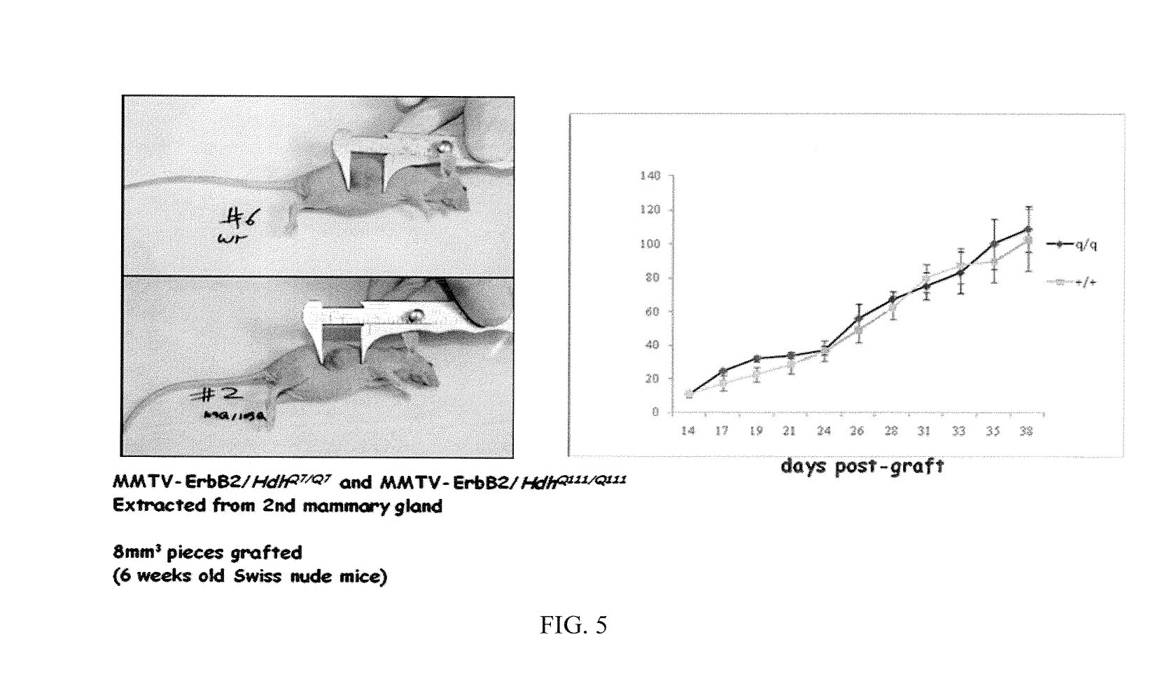

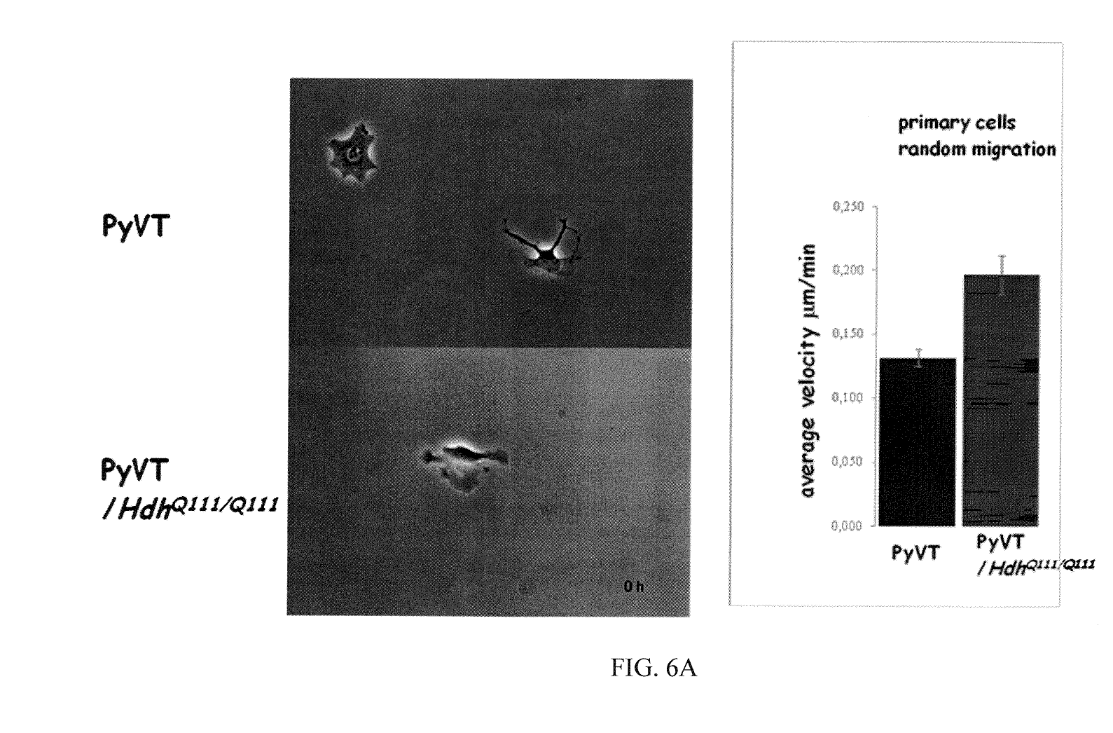

The inventors next evaluated the growth of tumors in wild-type and HD context by grafts experiments. Primary solid-tumor isolates from MMTV-PyVT; Hdh^{Q7/Q7} and MMTV-PyVT; Hdh^{Q111/Q111} were transplanted in immunodeficient mice and tumor size was measured as a function of time following transplantation. In both wild-type and HD background, the primary tumors grew linearly at similar rate (FIG. 5) showing that the faster appearance of tumors in HD models is not due to accelerated growth of the tumors.

PolyQ-Huntingtin Increases Breast Cancer Cell Motility, Invasiveness and Metastasis

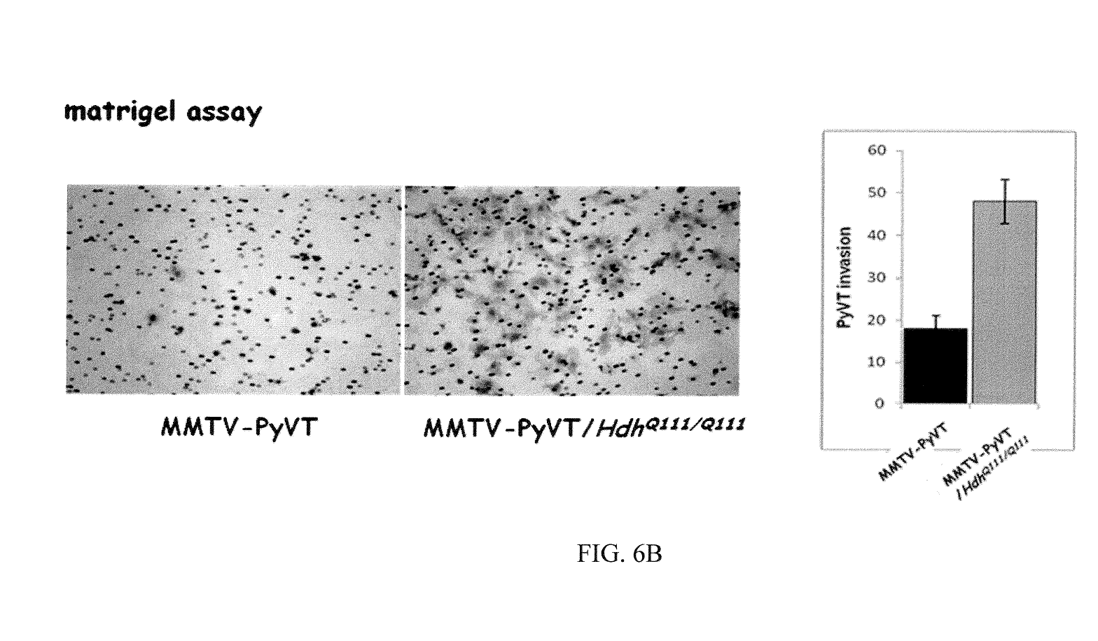

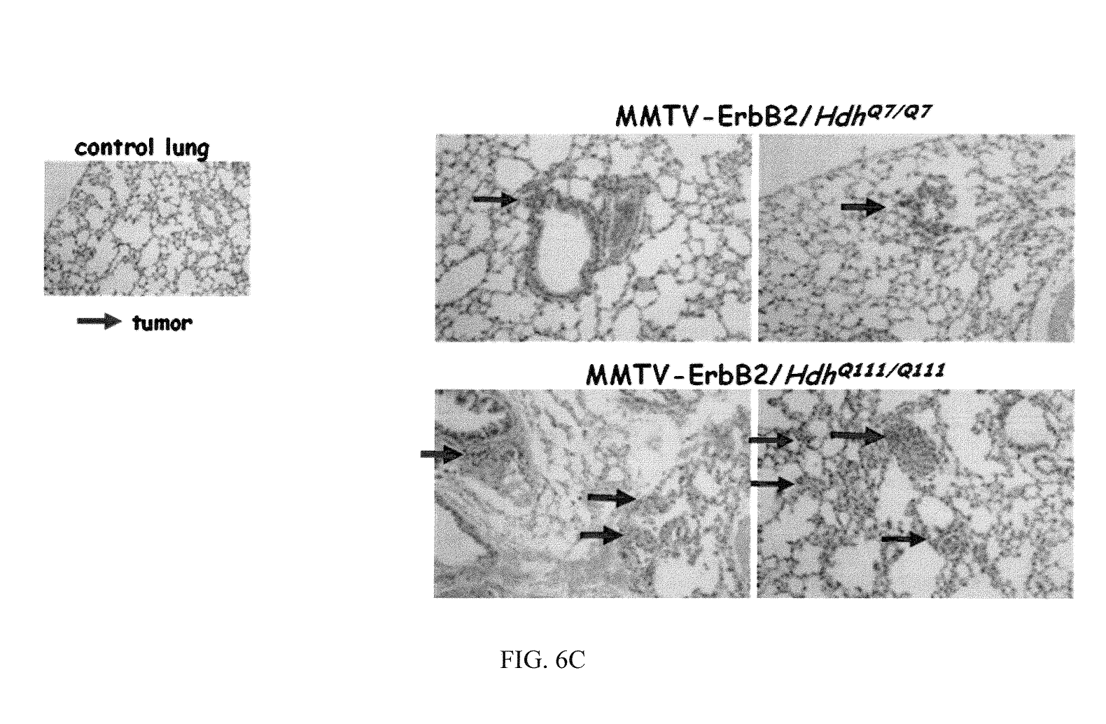

The inventors next derived primary tumors cells from MMTV-pyVT; Hdh^{7/Q7}/Q⁷ and MMTV-PyVT; Hdh^{Q111/Q111} tumors and performed random cell migration assays (FIG. 6A). Cells expressing the polyQ form of huntingtin moved faster than the control cells. The inventors also tested how polyQ huntingtin might alter cancer cell invasion in matrigel invasion chambers (FIG. 6B). In this assay, the primary cells derived from MMTV-PyVT; Hdh^{Q111/Q111} tumors show an increase in the number of cells which were able to invade through matrigel chambers compared to primary cells derived from MMTV-PyVT; Hdh^{Q7/Q7} tumors.

Based on the findings that polyQ-huntingtin leads to increased invasiveness, the inventors next examined metastasis to the lungs in immunodeficient mice grafted with primary solid-tumor isolates from MMTV-ErbB2; Hdh^{Q7/Q7} and MMTV-ErbB2; Hdh^{Q111/Q111} (FIG. 6C). Thirty-eight days after the grafts, lungs were dissected, sectioned and stained with hematoxylin and eosin. In animals grafted with MMTV-ErbB2; Hdh^{Q111/Q111} tumors, the metastasis was increased as compared to animals grafted with MMTV-ErbB2; Hdh^{Q7/Q7} tumors.

Thus, cells expressing polyQ-huntingtin are more motile and invasive than control tumor cells expressing wild-type huntingtin and this leads to increased metastasis in vivo in the polyQ context.

HD Patients Develop More Aggressive Forms of Breast Cancer than Non HD Patients

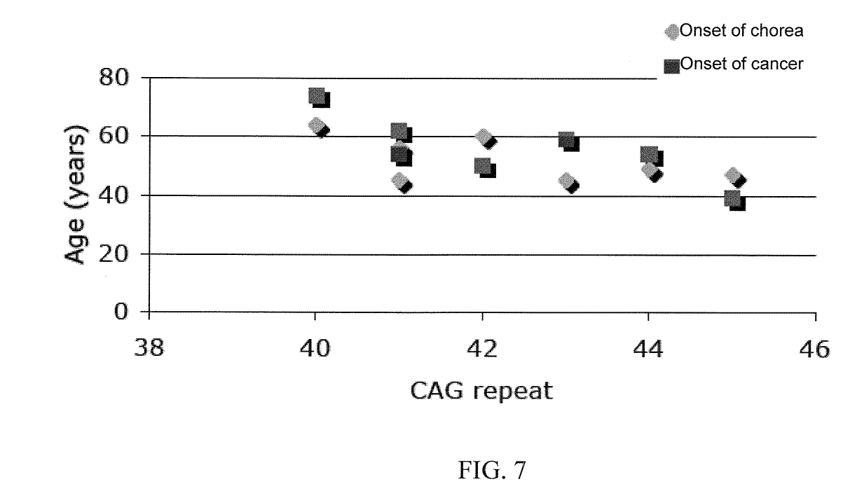

To establish the physiological relevance of the inventors' findings, they identified nine HD patients with breast cancer. The examination of their medical records revealed that they developed more aggressive forms of cancer. Indeed, the majority of them were grade III with metastasis (data not shown). In HD, there is an inverse correlation between the length of the CAG repeat and the age of onset with long expansions corresponding to juvenile forms of the disease (Young, 2003). Here, the inventors observed a striking correlation between the age of cancer onset and the polyglutamine length (FIG. 7). Together, these observations are in agreement with the mouse models data, showing that the abnormal polyQ expansion causing HD leads to more severe progression of breast cancer.

In conclusion, these results demonstrate that polyQ expansion in htt increases tumorigenesis and metastasis capacity.

### Example 3

### HTT and Prostate Cancer

Materials and Methods

siRNA and transfections—A small hairpin RNA construct against htt (shHtt) was used. The hairpin region of shHtt recognizes a region within exons 8-9 (AGCTTTGATGGATTCTAATCTCTTGAAATTAGAATCCATCAAAGCT, SEQ ID NO: 4). The shLuc construct is identical apart from its hairpin recognizing a sequence within the firefly luciferase gene rather than a sequence within htt.

Transfections were performed as described in example 1.

Cell line—The LNCaP human prostate cell line was used. The LNCaP cell line was established from a metastatic lesion of human prostatic adenocarcinoma.

Cell motility—Random migration assays were performed as described in example 1.

Results

shHtt has been used to decrease levels of htt in LNCaP human prostate cell line as described in example 1. The migration of these cells was then assessed (random migration assays) and compared to control cells (LNCap cells transfected with shLuc). It was observed that decreasing htt level induces an increased capacity of cells to move.

This demonstrates that htt is involved in motility of prostate cancer cells and that the dysregulation of this function participates to the metastasis and invasive capacities of these cells.

### REFERENCES

Anne, S. L., et al. (2007). J Neurosci 27, 7318-7328.
Bernstein, E., et al. (2001). Nature 409(6818): 363-6.
Elbashir, S. M., et al. (2001). Embo J 20(23): 6877-88.
Fanning and Symonds (2006) RNA Towards Medicine (Handbook of Experimental Pharmacology), ed. Springer p. 289-303.
Fitamant J. et al., (2008) Proc Natl Acad Sci USA 105, 4850.
Guy, C. T., et al. (1992). Mol Cell Biol 12, 954-961.
Humbert S, et al (2002) Dev Cell 2:831-837.
Leavitt B R, et al. (2001) Am J Hum Genet. 68(2):313-24.
Martindale D, et al. (1998) Nat Genet. 18(2):150-4.
Morton, C. L., and Houghton, P. J. (2007). Nat Protoc 2, 247-250.
Muller, W. J., et al. (1988). Cell 54, 105-115.
Muramatsu and Kincaid, (1996), BBRC, 218, 466-472.
Musaro et al, (1999), Nature, 6744, 581-585.
Nishimura and Tanaka, (2001), J. Biol. Chem., 276, 19921-19928.
Rigamonti D, et al. (2000) J. Neurosci. 20(10):3705-13.
Saudou, F., et al. (1998) Cell 95, 55-66.
Sharp A H and Ross C A. (1996) Neurobiol Dis. 3(1):3-15.
Shibasaki et al, (1996), Nature, 6589, 370-373.
Sørensen S A, et al. (1999) Cancer. 86(7):1342-6.
Taddei, I., et al. (2008). Nat Cell Biol 10, 716-722.
Wang G H, et al. (1999) Neuroreport. 10(12):2435-8.
Wang et al, (1999), Science 284, 339-343.
Wheeler, V. C., et al. (2002). Hum Mol Genet. 11, 633-640.
Yamashita, (2000), J. Exp. Med., 191, 1869-1880.
Young, A. B. (2003). J Clin Invest 111, 299-302.
Zala, D., et al. (2008). Hum Mol Genet. 17, 3837-3846.
Zamore, P. D., et al. (2000). Cell 101(1): 25-33.
Zeitlin S, et al. (1995) Nat Genet. 11(2):155-63.
Zhu et al. (2000) J. Biol. Chem., 275, 15239-15245.

We claim:

1. A method for diagnosing or detecting a cancer in a subject, wherein the method comprises the step of determining the cellular level of phosphorylated form of huntingtin in a sample from said subject, a low cellular level of phosphorylated huntingtin indicating that said subject suffers from a cancer.

2. The method according to claim 1, wherein the method further comprises the step of comparing the cellular level of phosphorylated huntingtin to a reference cellular level

3. The method according to claim 2, wherein the reference cellular level is the cellular level of phosphorylated huntingtin in a normal sample.

4. A method for predicting, prognosing or monitoring clinical outcome of a subject affected with a cancer, wherein the method comprises the step of determining (i) the expression level of huntingtin or (ii) the cellular level of phosphorylated huntingtin, in a cancer sample from said subject or (iii) the number of glutamine residues on the poly-Q expansion of huntingtin in a sample from said subject, a low expression level of huntingtin, a low cellular level of phosphorylated huntingtin or a poly-Q expansion comprising more than 20 glutamine residues, being indicative of a poor prognosis.

5. The method according to claim 4, wherein the method further comprises the step of comparing the expression level of huntingtin or the cellular level of phosphorylated huntingtin to a reference expression level.

6. The method according to claim 4, wherein a poly-Q expansion comprising more than 35 glutamine residues is indicative of a poor prognosis.

7. The method according to claim 4, wherein a poor prognosis is a decreased patient survival and/or an early disease progression and/or an increased metastasis formation.

8. A method for selecting a subject affected with a cancer for an antitumoral therapy or determining whether a subject affected with a cancer is susceptible to benefit from an antitumoral therapy, wherein the method comprises the step of determining (i) the cellular level of phosphorylated huntingtin or (ii) the expression level of huntingtin in a cancer sample from said subject or (iii) the number of glutamine residues on the poly-Q expansion of huntingtin in a sample from said subject, a low cellular level of phosphorylated huntingtin, a low expression level of huntingtin or a poly-Q expansion comprising more than 20 glutamine residues indicating that an antitumoral therapy is required.

9. A method for selecting, identifying or screening a compound useful for treating a subject having cancer, comprising the selection or identification of a compound capable of increasing the expression level and/or the phosphorylation of huntingtin.

10. The method according to claim 9, wherein said method comprises:
a) providing a huntingtin protein or a fragment thereof of at least 50 consecutive amino acids and comprising at least one phosphorylated residue selected from the group consisting of S421, S535, S1181, S1201, S2076, S2653 and S2657;
b) providing a compound dephosphorylating at least one phosphorylated residue comprised in htt protein or the fragment thereof provided in step a);
c) contacting a candidate compound with said huntingtin protein or fragment thereof and said dephosphorylating compound; and
d) selecting the candidate compound that inhibits the dephosphorylation of at least one phosphorylated residue comprised in htt protein or the fragment thereof provided in step a) by dephosphorylating compound.

11. The method according to claim 9, wherein said method comprises:
a) contacting a candidate compound with a cell expressing a huntingtin protein and comprising a kinase which phosphorylates huntingtin at a position selected from the group consisting of S421, S535, S1181, S1201, S2076, S2653 and S2657, and a compound dephosphorylating the phosphorylated residue at selected position;
b) assessing the amount of huntingtin phosphorylated and/or the amount of huntingtin which is not phosphorylated; and
c) selecting the candidate compound that increases the phosphorylation of huntingtin at selected position in comparison with a control cell which has not been contacted with the candidate compound.

12. The method according to claim 9, wherein said method comprises:
a) contacting a candidate compound with a cell expressing a huntingtin protein;
b) assessing the amount of huntingtin expressed in said cell; and
c) selecting the candidate compound that increases the expression of huntingtin in comparison with a control cell which has not been contacted with the candidate compound.

13. A method for treating cancer in a subject, comprising administering a therapeutically effective amount of a compound increasing the cellular level of the phosphorylated form of huntingtin.

14. The method according to claim 13, wherein the huntingtin protein is phosphorylated at one or several positions selected from the group consisting of S421, S535, S1181, S1201, S2076, S2653 and S2657.

15. The method according to claim 14, wherein the huntingtin protein is phosphorylated at position S421.

16. The method according to claim 13, wherein said compound is selected from the group consisting of huntingtin protein and a biologically active fragment thereof, huntingtin protein comprising the mutation S241D and a biologically active fragment thereof, and a nucleic acid encoding thereof.

17. The method according to claim 13, wherein said compound inhibits the dephosphorylation of huntingtin.

18. The method according to claim 17, wherein said compound is a calcineurin inhibitor or a compound inhibiting the interaction between calcineurin and huntingtin.

19. The method according to claim 18, wherein the calcineurin inhibitor is a nucleic acid molecule interfering specifically with calcineurin expression, preferably a RNAi, an antisense nucleic acid or a ribozyme.

20. The method according to claim 18, wherein the calcineurin inhibitor is a dominant-interfering form of calcineurin.

21. The method according to claim 13, wherein said compound increases the phosphorylation of huntingtin.

22. The method according to claim 13, wherein said cancer is an invasive cancer and/or a cancer capable of metastasis.

23. The method according to claim 13, wherein the cancer is selected from the group consisting of leukemia, lymphoma, melanoma, lung cancer, bowel cancer, colon cancer, rectal cancer, colorectal cancer, brain cancer, liver cancer, pancreatic cancer, breast cancer, prostate cancer, testicular cancer and retinoblastoma.

24. The compound according to claim 23, wherein the cancer is breast cancer or prostate cancer.

25. The compound according to claim 13, wherein the subject is a human, preferably a human not affected with Huntington's disease.
